(19) United States
(12) Patent Application Publication
(10) Pub. No.: US 2025/0341524 A1
(43) Pub. Date: Nov. 6, 2025

(54) FLUORESCENTLY-LABELED F-ACTIN PROTEIN BIOSENSORS AND METHODS OF HIGH-THROUGHPUT DRUG DISCOVERY

(71) Applicant: ARIZONA BOARD OF REGENTS ON BEHALF OF THE UNIVERSITY OF ARIZONA, Tucson, AZ (US)

(72) Inventor: Brett Colson, Tucson, AZ (US)

(21) Appl. No.: 17/793,319

(22) PCT Filed: Jan. 20, 2021
(86) PCT No.: PCT/US21/14142

§ 371 Date: Jul. 15, 2022

Related U.S. Application Data
(60) Provisional application No. 62/963,298, filed on Jan. 20, 2020.

(51) Int. Cl. G01N 33/58

(52) U.S. Cl. CPC G01N 33/582; G01N 2440/14; G01N 2500/02

(57) ABSTRACT

Protein biosensors for drug discovery, such as a high-throughput assay utilizing TR-F to detect the properties of binding proteins (e.g., actin-binding proteins, ABPs) using a fluorescence lifetime plate reader and fluorescent probes. In particular, the present invention is a new use of TR-F technique to rapidly evaluate binding of F-actin (− or +Tm) with cMyBP-C in solution. Changes in labeled actin fluorescence lifetime due to cMyBP-C phosphorylation and/or HCM mutations correlated with binding measured by traditional cosedimentation. The present invention features methods for HTS for identifying molecules that modulate cMyBP-C or cMyBP-C-actin complex.

### CROSS REFERENCE

This application claims benefit of U.S. Provisional Application No. 62/963,298, filed Jan. 20, 2020, the specification(s) of which is/are incorporated herein in their entirety by reference.

### STATEMENT REGARDING FEDERALLY SPONSORED RESEARCH OR DEVELOPMENT

This invention was made with government support under Grant No. R00 HL122397 and R01 HL141564 awarded by National Institutes of Health. The government has certain rights in the invention

### REFERENCE TO A SEQUENCE LISTING

Applicant asserts that the information recorded in the form of an Annex C/ST.25 text file submitted under Rule 13ter.1(a), entitled UNIA_19_20_Sequencing_Listing_ST25, is identical to that forming part of the international application as filed. The content of the sequence listing is incorporated herein by reference in its entirety

### BACKGROUND OF THE INVENTION

### Field of the Invention

The technology of the present invention features protein biosensors for drug discovery. In particular, the present invention features a high-throughput assay utilizing time-resolved fluorescence (TR-F i.e., fluorescence lifetime)) to detect the properties of binding proteins (e.g., actin-binding proteins, ABPs) using a fluorescence lifetime plate reader and fluorescent probes attached to F-actin at Cys374. In particular, the present invention is a new use of TR-F technique to rapidly evaluate binding of F-actin (− or +tropomyosin (Tm)) with cardiac myosin binding protein C (cMyBP-C) in solution. Changes in labeled actin fluorescence lifetime due to cMyBP-C phosphorylation and/or hypertrophic cardiomyopathy (HCM) mutations correlated with binding measured by traditional cosedimentation. The present invention features methods for high throughput screening (HTS) for identifying molecules that modulate cMyBP-C or cMyBP-C-actin complex. The assays of the present invention may also identify compounds that simply bind/disrupt F-actin alone.

### Background Art

Properties of F-actin binding to actin-binding proteins (ABPs) are typically evaluated by cosedimentation assays, which are time-consuming, involve multiple steps, and are limited in the number that can be done at one time.

CN10929817A (Jiangsu Meike Medical Technology): Cardiac myosin binding protein C (cMyBP-C based on immunomagnetic beads) time-resolved fluoroimmunoassay kit. The invention provides a cardiac myosin binding protein C (cMyBP-C) time-resolved fluoroimmunoassay kit based on immunomagnetic beads. JP2008516607A (Carnegie Institute of Washington): Development of high sensitivity FRET sensor and its use. The technology describes intramolecular biosensors that include a ligand binding domain fused to a donor and a fluorescent moiety that allows detection and measurement of fluorescence resonance energy transfer upon ligand binding. CN109444431A (Zhengzhou Autobio Diagnostics): A kind of quantitative detecting method and detection kit of cardiac myosin binding protein C. A quantitative detection method and kit of cardiac myosin binding protein C. The technology involves antibody coupling, chemiluminescence, and specific binding. U.S. Pat. No. 8,431,356B2: FRET assays for sarco/endoplasmic reticulum calcium ATPase and phospholamban. The technology is an assay using FRET that is optimized for high-throughput screening for identifying small molecules that modulate SERCA or the SERCA-PLB complex.

Red-Shifted FRET Biosensors for High-Throughput Fluorescence Lifetime Screening. Development of FRET biosensors with red-shifted fluorescent proteins. The fluorescent proteins were fused to specific sites on the human cardiac calcium pump (SERCA2a) for detection of structural changes to small-molecule effectors. High-Throughput screen, using time-resolved FRET, yields actin-binding compounds that modulate actin-myosin structure and function. Article discusses FRET based assay to detect small-molecule modulators of actin-myosin structure and function.

Relevant commercial kits include: The RapidFire High-Throughput MS systems (Agilent) is a Mass Spectrometry system that significantly increases the throughput of LC/MS analyses. A Co-Sedimentation Assay for the Detection of Direct Binding to F-Actin Protocol is used for the detection of direct binding to F-actin.

### BRIEF SUMMARY OF THE INVENTION

The present invention features a simple, quantitative F-actin binding assay that can be scaled up, e.g., to a 384-well or 1536-well plate format (or other sizes), useful for high-throughput screening (HTS) of drugs targeting ABP's in human disease. In particular, the present invention uses time-resolved fluorescence (TR-F) analyzed by fluorescence lifetime plate reader instrumentation to monitor fluorescently labeled actin binding to an ABP, e.g., cardiac myosin binding protein-C (cMyBP-C) (e.g., fluorescent probes attached to F-actin at Cys374) and detect changes in actin binding upon physiological changes, phosphorylation, and/or mutation of the ABP.

This assay may be particularly useful for heart disease, the leading cause of death in the US. An example of how TR-F is used to quantitate binding of actin to cardiac myosin protein-C (MyBP-C) and how this binding is affected by physiological conditions and compounds is described herein. The present invention can lead to potential therapeutic discoveries to treat cardiomyopathy (e.g., hypertrophic cardiomyopathies, dilated cardiomyopathies, etc.) and heart failure (e.g., heart failure with preserved ejection fraction (HFpEF), heart failure with reduced ejection fraction (HFrEF), etc.). As described herein, the technology has been tested and shown to get similar results to the current methods used (e.g., co-sedimentation). Applications of this technology include drug discovery, cosedimentation analysis, high-throughput assay, protein biosensors, and cardiac disease. The advantages of the present invention comprise faster detection and high functionality.

It is an objective of the present invention to provide methods of use and kits as means for using TR-F that allow for quantitating protein binding changes, particularly for actin and ABPs, as well as for identifying or screening compounds that alter actin-cMyBP-C interactions as specified in the independent claims. Embodiments of the invention are given in the dependent claims. Embodiments of the present invention can be freely combined with each other if they are not mutually exclusive.

The present invention features a method of using TR-F and fluorescent protein biosensors to quantitate protein binding in solution. In preferred embodiments, the method is used to quantitate actin protein binding in solution, e.g., actin-cMyBP-C binding. In preferred embodiments, the method comprises first operably connecting (e.g., labelling) a first protein (e.g., actin, actin-tropomyosin complex) with a fluorescent probe suitable for TR-F. Non-limiting examples of the fluorescent probe comprise IAEDANS, IAANS, CPM, IANBD, FMAL, 5-IAF, and the Alexa Fluor dye series (e.g., Alexa Fluor 568, Alexa Fluor 488, Alexa Fluor 532). The first protein labelled with the fluorescent probe is then contacted (e.g., bound) with the second protein (e.g., cMyBP-C) in solution. Time Resolved Fluorescence (TR-F) lifetime is then measured when the fluorescence is effectuated and can be measured using lifetime fluorescent plate readers. Fluorescence lifetime is the time that a fluorescent probe is in the excited state and decays down to the ground state. Protein binding or contact is quantitated using the measured fluorescence lifetime.

The present invention also features a method of identifying a molecule that modulates actin binding to ABPs. In preferred embodiments, the method comprises first operably connecting (e.g., labelling) actin or actin-tropomyosin complex with a fluorescent probe suitable for TR-F. Non-limiting examples of the fluorescent probe comprise IAEDANS, IAANS, CPM, IANBD, FMAL, 5-IAF, and Alexa Fluor dyes (e.g., Alexa Fluor 568, Alexa Fluor 488, Alexa Fluor 532). The actin that is operably connected/labelled to the fluorescent probe is then contacted including bound to an ABP in solution. TR-F fluorescence lifetime is measured when fluorescent probe labelled-actin contacts or binds ABP in the presence and absence of a molecule. The fluorescence lifetime is the time that a fluorescent probe is in the excited state and decays down to the ground state. Actin-ABP protein binding is then quantitated using the measured fluorescence lifetime in the presence and absence of molecules (see Equation 1). The molecule is identified as a modulator of actin-ABP binding when a change occurs in TR-F fluorescence lifetime in presence of the molecule as compared to absence of the molecule.

The present invention also features a kit comprising a means for identifying a molecule that modulates actin binding to ABPs. In preferred embodiments, the kit comprises a means for operably connecting (e.g., labelling) actin or actin-Tm complex with a fluorescent probe suitable for TR-F. Non-limiting examples of the fluorescent probe comprise IAEDANS, IAANS, CPM, IANBD, FMAL, 5-IAF, and Alexa Fluor dyes (e.g., Alexa Fluor 568, Alexa Fluor 488, Alexa Fluor 532). The kit provides a means for contacting or binding the actin that is operably connected/labelled to the fluorescent probe to an ABP in solution. The kit also provides a means for performing TR-F. TR-F fluorescence lifetime is measured when fluorescent probe labelled-actin contacts or binds ABP in the presence and absence of a molecule. The fluorescence lifetime is the time that a fluorescent probe is in the excited state and decays down to the ground state. Actin-ABP protein binding is then quantitated using the measured fluorescence lifetime in the presence and absence of molecule and identifying the molecule as a modulator of actin-ABP binding when a change occurs in TR-F fluorescence lifetime in presence of the molecule as compared to absence of the molecule.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIGS. 1A and 1B show lifetime changes of five fluorescent dyes attached to actin at Cys374 upon binding to C0-C2. Either 1 or 5 μM of fluorescently labeled-actin was mixed with either 0, 5, or 20 μM of unlabeled C0-C2. From left to right bars represent actin alone (1), 5 μM of unphosphorylated C0-C2 (2), 20 μM of unphosphorylated C0-C2 (3), 5 μM of phosphorylated C0-C2 (4), and 20 μM of phosphorylated C0-C2 (5). FIG. 1A shows the fluorescence lifetime and FIG. 1B shows the relative change in lifetime.

FIGS. 2A, 2B, 2C, 2D, 2E, 2F, 2G, and 2H show TR-F binding assays from actin (thin lines) and actin-Tm (thick lines) compared to cosedimentation. Unphosphorylated C0-C2 (solid lines) and phosphorylated C0-C2 (dotted lines) were assayed. FIG. 2A shows TR-F using IAEDANS-actin (thin lines). FIG. 2B shows TR-F using IAEDANS-actin-Tm (thick lines). FIG. 2C shows TR-F using IAEDANS-actin and IAEDANS-actin-Tm showing 0-5 μM C0-C2 added. FIG. 2D shows cosedimentation using actin. FIG. 2E shows cosedimentation using actin-Tm. FIG. 2F shows cosedimentation using IAEDANS-actin and IAEDANS-actin-Tm showing 0-5 μM C0-C2 added. Arrows in FIGS. 2A-2F indicates the C0-C2 concentrations to be used in future screens (thin arrows, 2.5 μM for actin alone and thick arrows, 1.25 μM for actin-Tm). FIG. 2G shows a linear correlation plot of unphosphorylated C0-C2 binding measured by TR-F (change in lifetime) and cosedimentation ([mol. bound C0-C2]/[actin]) for actin (R²=0.96) with each assay readout normalized to 1 (at 20 μM C0-C2). FIG. 2H shows linear correlation plot in the presence of Tm, under conditions of FIG. 2G, for unphosphorylated C0-C2 binding to Actin-Tm (R²=0.87). Refer to Table 1 for statistical analysis of fitted binding properties for curves. Data are provided as mean±SE (n>4).

FIGS. 3A, 3B, and 3C show buffer conditions optimization of the TR-F assay for C0-C2 binding to IAEDANS-actin and IAEDANS-actin-Tm. Reduction in lifetimes (% decrease) for either 1 μM IAEDANS-labeled actin or actin-Tm binding to the indicated concentrations of C0-C2 that was not phosphorylated (bars 1, 2 & 3) or phosphorylated by PKA (+PKA, bars 4, 5, & 6). Note the first “bar” left of the 6 colored bars in each group shows 0% decrease in lifetime with baseline error bars for actin, or actin-Tm, alone (i.e., 0% decrease relative to itself), bars from left to right represent 1.25 μM C0-C2, 5 μM C0-C2, 20 μM C0-C2, 1.25 μM C0-C2+PKA, 5 μM C0-C2+PKA, and 20 μM C0-C2+PKA: FIG. 3A shows the binding buffers at varying pH and either 100 mM KCl or NaCl; FIG. 3B shows three (3) concentrations of KCl in the binding buffer; and FIG. 3C shows different ratios of Actin:Tm. In FIG. 3C, for unphosphorylated C0-C2 significant differences comparing actin alone to Actin:Tm are indicated: $p<0.05. In FIG. 3A-3C, significant differences between minus and plus PKA are indicated: *p<0.05, #p<0.005. Data is provided as mean±SE (n>4).

FIGS. 4A and 4B show Actin:Tm ratios. FIG. 4A shows relative staining intensity of actin and actin-Tm in SDS-PAGE gels stained with Coomassie blue. 1 μg actin from 2 separate preps (lanes 1-6) and 1 μg Tm lanes (7-9) were compared as were 0.2 μg actin (lanes 10-15) and 0.2 μg Tm (lanes 16-18). The concentrations of actin and Tm were determined using their extinction coefficients. For actin, Abs 290 0.1% (=1 g/l) was 0.63. For Tm Abs 280 0.1% (=1 g/l) was 0.274. The average actin/Tm staining intensity ratio was 1.55. FIG. 4B shows mixtures of 3.5:1 Actin:Tm were made (Total, first 6 lanes). Following centrifugation (TLA 100 rotor, 100K rpm, 30 min, 4° C.) actin and bound (cosedimenting) Tm in the pellet were examined (Pellet, lanes 7-12). Supernatant (unbound Tm) was also examined (Supernatant, lanes 13-18). The relative intensities of the actin and Tm bands was 3:1 for the Total and 7.5:1 for the Pellet following correction for staining differences determined in A and differences in molecular weights (actin, 42,000 Daltons, and Tm dimer, 65,300 Daltons). Total and Supernatant can be directly compared as the same volume was examined for each. 40% of the input Tm remained unbound indicating that there was excess Tm in the mixture. All of the actin was found in the pellet.

FIGS. 5A, 5B, 5C, 5D, and 5E show an example of Z′ score calculation for time-resolved fluorescence (TR-F) of IAEDANS-labeled F-actin binding to unphosphorylated and phosphorylated C0-C2. FIG. 5A shows fluorescence waveform of IAEDANS-labeled F-actin and the same together with 20 μM C0-C2 (both normalized to their maximum intensity values) and the instrument response function (IRF). Inset box highlights relative fluorescence intensity ˜1/e magnified to show difference in TR-F lifetimes of actin and actin+C0-C2. This is expanded further in FIG. 5B. FIG. 5B shows a magnified view of lifetime differences from inset box in FIG. 5A. The actual lifetimes (around 17.5 ns, shown in C-E) are the times shown on the x-axis to reach 1/e of the peak intensity (around 22.5 ns) minus the time to reach peak intensity (around 5 ns in the convoluted fluorescence waveform, see peak in FIG. 5A). FIG. 5C shows lifetimes measured in a 384-well plate containing 60 wells each of 1 μM IAEDANS-actin alone, actin plus 2.5 μM C0-C2, or actin plus 2.5 μM PKA-treated C0-C2. FIG. 5D compares actin alone to actin plus C0-C2. FIG. 5E compares actin plus C0-C2 vs. actin plus PKA-treated C0-C2. For FIGS. 5D and 5E, horizontal solid lines indicate 3 standard deviations (3×SD) of the mean lifetime (dotted line). Z′score is defined as the difference between 3×SD (a) divided by the difference in the mean signal (b) in FIG. 5D and FIG. 5E. While comparisons made in FIG. 5D and FIG. 5E, having no overlap at 3×SD, are clearly significantly different, note that even the difference between actin alone and actin bound to phosphorylated C0-C2 (FIG. 5C) is also significant (p<0.0001).

FIG. 6A-6C shows MyBPC organization and C0-C2 mutants tested. FIG. 6A shows full-length cMyBP-C domains C0 through C10. Ig-like domains shown as circles and Fn3-like domains as hexagons. FIG. 6B shows C0-C2 domains containing P/A linker and phosphorylatable M-domain are shown. Sequence of M-domain (SEQ ID NO: 1) and locations of HCM mutations tested for binding. PKA phosphorylatable serines are denoted by #, PKA recognition sequences are indicated with underlines, and HCM mutation sites are denoted by *. Helix residues in the tri-helix bundle are indicated with thick underlines. Structure insets: tri-helix bundle (PDB ID: 5K6P) containing L352P and E334K mutations and C1 (PDB ID: 6CXI) showing the RASK loop between adjacent beta-strands that interact with Tm. FIG. 6C shows that C0-C1 is a deletion of the M-domain and C2.

FIG. 7 shows TR-F binding curves of WT C0-C2, L352P, E334K, and C0-C1 on IAEDANS-actin. TR-F of IAEDANS-actin binding to 0-20 μM C0-C2 and C0-C1. Data are provided as mean±SE (n>4).

FIG. 8A-8D shows the effects of WT and R282W HCM mutant on phosphorylation-modulated binding to actin at submaximal phosphorylation by PKA. FIG. 8A shows TR-F of IAEDANS-actin incubated with increasing concentrations of C0-C2 (WT and R282W) either unphosphorylated (solid lines) or phosphorylated (dashed lines) using 7.5 ng PKA/μg C0-C2. Curved in descending order are WT C0-C2 (solid), R282W C0-C2 (solid), R282W+PKA (dashed), and WT+PKA (dashed). For FIG. 8B-8D shows the effects of HCM mutant R282W on C0-C2 phosphorylation were tested over a range of PKA levels (0-5 ng PKA/μg C0-C2). FIG. 8B shows Sypro Ruby (total protein; top bands) and Pro-Q Diamond (phosphorylated protein; bottom bands) stains of SDS-PAGE. FIG. 8C shows relative phosphorylation levels of WT and R282W (normalized to the ratio of the Pro-Q Diamond/Sypro Ruby intensities for WT C0-C2 at 5 ng PKA/μg C0-C2). Phosphorylation levels of R2828W are significantly different (p<0.00006) from WT for all concentrations of PKA. FIG. 8D shows WT and R282W effects on IAEDANS-actin lifetime change as a function of PKA concentration. At intermediate phosphorylation levels (0.5 and 1.5 ng PKA/μM C0-C2) binding to actin detected by TR-F is significantly different between WT and R282W (*p<0.003). Average data are provided as mean±SE (n>4).

FIGS. 9A, 9B, 9C and 9D shows the effects of WT and Tm-binding mutants on binding to actin-Tm and actin. Effects of WT and Tm-binding mutants on actin-Tm (thick lines) and actin (thin lines) TR-F were tested. Tm-binding mutants reverse charges (EASE; R215E/K218E) or introduce additional positive charges (RRKK; A216R/S217K) in the Tm binding loop 215-218, RASK, of C0-C2. FIG. 9A shows WT and R215E/K218E (“EASE”) effects on IAEDANS-actin-Tm and IAEDANS-actin for C0-C2 from 0 to 20 μM. FIG. 9B shows a zoom in on the lower concentration (0 to 5 μM) C0-C2 added in FIG. 9A. FIG. 9C-9D shows the same conditions as FIG. 9A-9B above but comparing WT and A216R/S217K (“RRKK”). For the Tm-binding mutant (charge reversal-EASE), apparent K_{d} changes trended toward significance (p=0.11) for actin-Tm but not for actin alone. For the positive Tm-binding mutant (additional positive charges-RRKK) apparent K_{d} changes were significant (p<0.05) for binding to both actin and actin-Tm. Refer to Table 4 for statistical analysis of fitted binding of fitted binding properties for curves and Table 2 for comparisons of binding at specific C0-C2 concentrations. Data are provided as mean±SE (n>4).

FIGS. 10A, 10B, 10C, 10D, 10E, 10F, 10G, and 10H show TR-F IAEDANS-actin and IAEDANS-actin Tm binding curves for C0-C2 mutants. TR-F measurements of the effects of mutant C0-C2 on the reduction (% decrease) in lifetimes are plotted for increasing concentration without (solid lines) and with PKA treatment (dotted lines). For comparison, curves for WT C0-C2 are included in each graph. FIG. 10A shows C0-C1/actin-Tm. FIG. 10B shows C0-C1/actin. FIG. 10C shows L352P/actin-Tm. FIG. 10D shows L352P/actin. FIG. 10E shows E334K/actin-Tm. FIG. 10F shows E334K/actin. FIG. 10G shows R282W/actin-Tm. FIG. 10 H shows R282W/actin. Arrows indicate the C0-C2 concentrations to be used in future screens (thick arrows, 1.25 μM for actin-Tm in FIGS. 10A, 10C, 10E, and 10G and thin arrows, 2.5 μM for actin alone in 10B, 10D, 10F, and 10H). Data is provided as mean±SE (n>4).

FIGS. 11A, 11B, 11C, 11D, 11E, 11F, 11G, and 11H show the effects of WT and Tm-binding mutants on phosphorylation-dependent binding to actin and actin-Tm. Effects Tm-binding mutants C0-C2 on actin-Tm and actin TR-F were tested with and without PKA treatment. Tm-binding mutants reverse charges (EASE; R215E/K218E) or introduce additional positive charges (RRKK; A216R/S217K) in the Tm binding loop 215-218, RASK, of C0-C2. For comparison, curves for WT C0-C2 are included in each graph. FIG. 11A shows WT and R215E/K218E (“EASE”) effects on IAEDANS-actin-Tm for unphosphorylated (solid lines) and phosphorylated (+PKA, dotted lines) C0-C2 from 0 to 20 μM cMyBP-C added. FIG. 11B shows the same conditions as FIG. 11A, except that IAEDANS-actin was used. FIGS. 11C and 11D show a zoom in on the lower concentrations (0 to 5 μM) cMyBP-C added in FIGS. 11A and 11B. FIGS. 11E and 11H show the same conditions as FIG. 11A-11D above but comparing WT (black lines) and A216R/S217K (“RRKK”). For the unphosphorylated Tm-binding mutant (charge reversal-EASE), apparent Kd changes trended toward significant (p=0.11) for actin-Tm but not for actin alone. The phosphorylated mutant did not fit well to a quadratic binding equation for either actin or actin-Tm (but did fit to a linear equation). For the unphosphorylated positive Tm-binding mutant (additional positive charges-RRKK) apparent Kd changes were significant (p<0.05) for binding to both actin and actin-Tm. For phosphorylated RRKK binding, changes in Kd did not reach significance. Refer to Table 4 for statistical analysis of fitted binding properties for curves. See Table 5 for comparisons of binding at specific substoichiometric C0-C2 concentrations. Arrows indicate the C0-C2 concentrations to be used in future screens (thick arrows, 1.25 μM for actin-Tm in FIGS. 11A, 11C, 11E, and 11G and thin arrows, 2.5 μM for actin alone in FIGS. 11B, 11D, 11F, and 11H). Data are provided as mean±SE (n>4).

FIGS. 12A, 12B, 12C, and 12D show comparison of TR-F and cosedimentation actin binding assays for C0-C2 mutants. Unphosphorylated and phosphorylated WT and 5 mutants of C0-C2 were assayed using IAEDANS-actin or IAEDANS-actin-Tm. FIG. 12A shows TR-F using 1.25 μM C0-C2. FIG. 12B shows cosedimentation using 1.25 μM C0-C2. FIG. 12C shows TR-F using 10 μM C0-C2. FIG. 12D shows cosedimentation using 10 μM C0-C2. *p<0.05 for comparisons with WT under the same conditions at substoichiometric binding levels (1.25 μM C0-C2, FIGS. 12A and 12B). In FIGS. 12A and 12B, reduction in binding due to phosphorylation was significant (p<0.005) in all cases with the exception of EASE C0-C2 binding to actin-Tm measured by TR-F. Data is provided as mean±SE (n>4).

FIGS. 13A-13B show cosedimentation assays for C0-C1 binding to actin-Tm and actin. C0-C1 was assayed and unphosphorylated C0-C2 (solid lines) and phosphorylated C0-C2 (dotted lines) are shown for reference. FIG. 13A shows cosedimentation using actin-Tm. FIG. 13B shows cosedimentation using actin. Cosedimentation using actin-Tm or actin was also measured at 40 μM C0-C1. This was used for determination of Kd and Bmax for C0-C1 curves (Table 4). Data are provided as mean±SE (n>4).

FIG. 14 shows the effects of R282W HCM mutant on phosphorylation-modulated binding to actin-Tm at submaximal phosphorylation by PKA. Effects of HCM mutant R282W on C0-C2 phosphorylation were tested over a range (0-5 ng PKA/μg C0-C2) of PKA levels. WT and R282W effects on IAEDANS-actin-Tm lifetime change (% decrease) as a function of PKA concentration. At intermediate phosphorylation levels (0.5 and 1.5 ng PKA/μM C0-C2) binding to actin or actin-Tm detected by TR-F is significantly increased in R282W (*p<0.003). Data are provided as mean±SE (n>4).

### DETAILED DESCRIPTION OF THE INVENTION

As used herein, the term “biosensor” refers to an analytical device, used for the detection of a chemical substance that combines a biological component with a physicochemical detector. The sensitive biological element, e.g. tissue, microorganisms, organelles, cell receptors, enzymes, antibodies, nucleic acids, etc., is a biologically derived material or biomimetic component that interacts with, binds with, or recognizes the analyte under study.

As used herein, the term “biosensor” may also refer to labeled-actin or labeled-actin in the presence of cMyBP-C. As an example in a screen, a control plate may be used to test labeled-actin alone against a compound library to identify compounds that interfere with just actin (these are undesirable hits); then, the test screen may be performed with a mixture of labeled-actin and cMyBP-C (C0-C2; e.g., 1 uM labeled actin+2 uM C0-C2 in each well plus the compound in DMSO) and these are the desirable hits.

As used herein, the term “fluorescent protein biosensor” refers to a biosensor comprising a protein and a fluorescent probe operably connected.

As used herein, the term “high throughput” refers to automation of experiments such that large scale repetition becomes feasible.

As used herein, time-resolved fluorescence refers to time-resolved fluorescence spectroscopy is an extension of fluorescence spectroscopy. Here, the fluorescence of a sample is monitored as a function of time after excitation by a flash of light. The time resolution can be obtained in a number of ways, depending on the required sensitivity and time resolution. In preferred embodiments, direct waveform recording is used.

As used herein, time-resolved fluorescence lifetime changes refers to the increase or decrease in the fluorescence lifetime of the fluorescence biosensor (e.g., fluorescently-labeled actin) due to the binding an ABP (e.g., cMyBP-C) or changes in the fluorescence lifetime of bound complex (e.g., labeled-actin-cMyBP-C) due to a perturbation including but not limited to: ABP concentration, phosphorylation, mutation or the addition of another molecule/compound.

As used herein “time-resolved fluorescence (TR-F) and fluorescence lifetime may be used interchangeably.

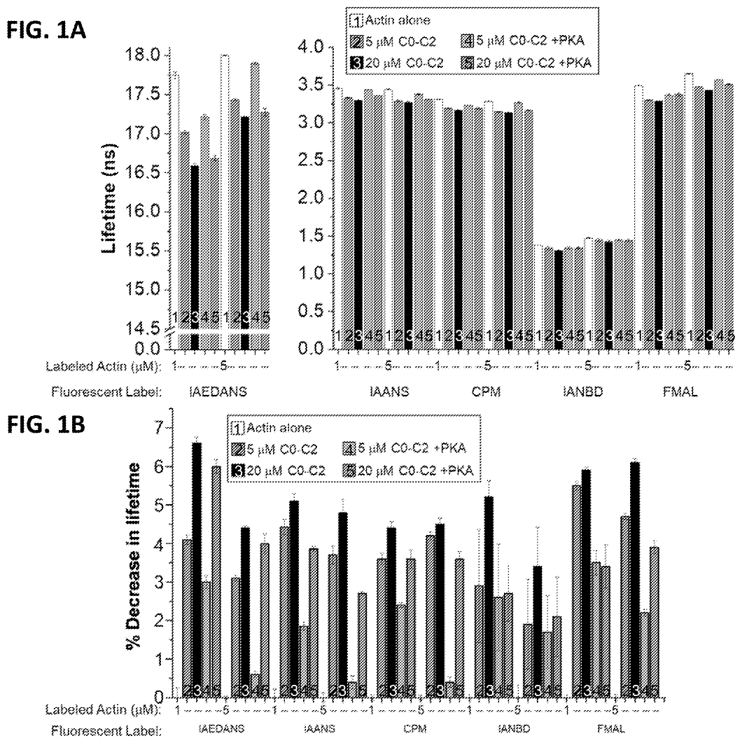

Referring now to FIGS. 1A-14, the present invention features methods and kits for using TR-F and a fluorescent protein biosensor to quantitate protein binding in solution, to quantitate actin protein binding in solution, and to identify a molecule that modulates actin binding to ABPs.

The present invention features a method of using time-resolved fluorescence (TR-F) and a fluorescent protein biosensor to quantitate protein binding in solution. In some embodiments, the method comprises labeling (operably connecting) a first protein with a fluorescent probe to generate a fluorescent protein biosensor suitable for TR-F. In some embodiments, the method comprises contacting including binding the first protein that is operably connected/labelled to the fluorescent probe to a second protein in solution. In further embodiments, the method comprises measuring TR-F fluorescence lifetime when the first protein contacts a second protein effectuating fluorescence. In some embodiments, the fluorescence lifetime is the time that a fluorescent probe is in the excited state and decays down to the ground state. In other embodiments, the method comprises quantitating protein contact including binding using the measured fluorescence lifetime.

The present invention may also feature a method of using time-resolve (TR-F) to quantitate actin protein binding in solution. In some embodiments, the method comprises labelling (operably connecting) actin with a fluorescent probe suitable for TR-F and contacting the fluorescent probe labelled actin to an actin-binding protein (ABP) in solution. In some embodiments, the method comprises measuring TR-F fluorescence lifetime when actin contacts ABP (in a region to effectuate) effectuating fluorescence. In some embodiments, the fluorescence lifetime is the time that a fluorescent probe is in the excited state and decays down to the ground state. In other embodiments, the method comprises quantitating actin-ABP binding using the measured fluorescence lifetime.

The present invention may further feature a method of identifying a molecule that modulated actin binding to actin binding proteins (ABPs) In some embodiments, the method comprises labelling (operably connecting) actin protein with a fluorescent probe suitable for time-resolved fluorescence (TR-F). In some embodiments, the method comprises contacting including binding actin that is operably connected/labelled to the fluorescent probe to an actin binding protein (ABP) in solution. In other embodiments, the method comprises measuring TR-F fluorescence lifetime when fluorescent probe labelled-actin contacts or binds ABP in the presence and absence of a molecule. In some embodiments, the fluorescence lifetime is the time that a fluorescent probe is in the excited state and decays down to the ground state. In further embodiments, the method comprises quantitating protein binding using the measured fluorescence lifetime in the presence and absence of molecule and identifying the molecule as a modulator of actin-ABP binding when a change occurs in TR-F fluorescence lifetime in presence of the molecule as compared to absence of the molecule.

Additionally, the present invention may feature a kit for identifying a molecule that modulated actin binding to actin binding proteins (ABPs). In some embodiments, the kit comprises a means for labelling (operably connecting) actin protein with a fluorescent probe suitable for time-resolved fluorescence (TR-F). In some embodiments, the kit comprises a means for contacting including binding actin that is operably connected/labelled to the fluorescent probe to an actin binding protein (ABP) in solution. In other embodiments, the kit comprises a means for measuring TR-F fluorescence lifetime when fluorescent probe labelled-actin contacts or binds ABP in the presence and absence of a molecule. In some embodiments, the fluorescence lifetime is the time that a fluorescent probe is in the excited state and decays down to the ground state. In some embodiments, the kit further comprises a means for quantitating protein binding using the measured fluorescence lifetime in the presence and absence of molecule and a means for identifying the molecule as a modulator of actin-ABP binding when a change occurs in TR-F fluorescence lifetime in presence of the molecule as compared to absence of the molecule.

The present invention may feature a method of identifying a test compound that modulates actin binding to actin-binding proteins (ABPs) or identifying a test compound that modulates an actin+actin-binding protein (ABP) complex or its microenvironment, wherein the method is suitable for high throughput screening (HTS). In some embodiments, the method comprises providing actin with a fluorescent probe suitable for time-resolved fluorescence (TR-F) and introducing actin with the fluorescent probe suitable for TR-F to an actin binding protein (ABP) in solution. In other embodiments, the method comprises measuring TR-F fluorescence lifetime when actin with the fluorescent probe suitable for TR-F contacts or binds ABP in the presence and absence of a test compound. In some embodiments, the fluorescence lifetime is the time that a fluorescent probe is in the excited state and decays down to the ground state. In further embodiments, the method comprises quantitating protein binding using the measured fluorescence lifetime in the presence and absence of the test compound and identifying the test compound as a modulator of actin-ABP binding when a change occurs in TR-F fluorescence lifetime in presence of the test compound as compared to absence of the test compound.

In preferred embodiments, the first protein (e.g., actin) and second protein (e.g., cMyBP-C) operably connect or bind in physiological solution. In some embodiments the actin and ABP contact including binding each other.

In other embodiments, a physiological change/perturbation, phosphorylation, and/or mutation of the second protein (e.g., phosphorylation or mutation of cMyBP-C) in proximity of operable connection between the first protein (e.g., fluorescently labelled actin) and second protein (e.g., cMyBP-C) affects a change in contact including binding of first protein. This affects or changes fluorescence lifetime and this change in fluorescence lifetime quantitates the change in contact including binding of the two proteins.

In some embodiments, a physiological change/perturbation, phosphorylation, and/or mutation of the ABP in proximity of operable connection between actin and ABP affects a change in binding of actin affecting/changing fluorescence lifetime, wherein change in fluorescence lifetime quantitates the change in binding.

In some embodiments, the first protein may comprise actin, globular actin (G-actin), fibrous-actin (F-actin), actin filament, actin-tropomyosin complex, or the regulated thin filament complex (F-actin, Tm, and the troponin complex of TnC, TnI, and TnT). In some embodiments, actin may comprise globular actin (G-actin), fibrous-actin (F-actin), actin filament, actin-tropomyosin complex, or thin filaments. In other embodiments, the second protein may comprise cardiac myosin binding protein-C (cMyBP-C), skeletal MyBP-C, or a fragment thereof, e.g., a fragment of cMyBP-C (e.g., C0-C2). In some embodiments, the ABP may comprise cardiac myosin binding protein-C (cMyBP-C), skeletal MyBP-C, or fragments thereof (e.g., C0-C2). In further embodiments, the second protein is any other actin-binding protein that binds near the probe on the actin at Cys374.

In certain embodiments, TR-F is performed using lifetime fluorescent plate readers. In preferred embodiments, TR-F is performed using lifetime fluorescent plate readers for high throughput screening (HTS). In other embodiments, TR-F is performed using a non-plate reader. In some embodiments, TR-F is performed using an instrument comprising a cuvette or in the chamber or a stopped-flow instrument.

In some embodiments, the fluorescent probes comprise thiol-reactive dyes containing a maleimide or iodoacetamide for conjugation with a cysteine on the protein. In other embodiments, the fluorescent probes comprise other chemistry groups (e.g., amine-reactive), affinity tags (e.g., His-tag) or peptides (e.g., Lifeact) conjugate with a non-cysteine residue (e.g., lysine or N-terminal amine) or peptide-binding region on the protein. In other embodiments, the fluorescent probes comprises a 355-532 nm excitation range. Without wishing to limit the present invention to any theories or mechanisms it is believed that red-shifted dyes excited towards the 532 nm help reduce interference with compound autofluorescence in screens.

In some embodiments, a fluorescent probe suitable for time-resolved fluorescence (TR-F) is any fluorescent dye described herein.

Non-limiting examples of the fluorescent probe comprise IAEDANS, IAANS, CPM, IANBD, FMAL, 5-IAF, and Alexa Fluor dyes (such as but not limited to Alexa Fluor 488, Alexa Fluor 532, and Alexa Fluor 568). In some embodiments, fluorescent probes with the biggest relative (%) change with C0-C2 binding and those with small errors and reproducibility are selected for the methods and kits described herein.

In other embodiments, the method and/or kit detects binding properties of the first protein by correlating time-resolved fluorescence lifetime with protein binding interactions in solution. In some embodiments, the method and/or kit is for screening physiological conditions or compounds that affect the second protein contact including binding to the first protein. In some embodiments, the method and/or kit detects binding properties of actin by correlating time-resolved fluorescence lifetime with actin-ABP binding interactions in solution. In other embodiments, the method and/or kit is for screening physiological conditions or compounds that affect the ABP binding to actin. In other embodiments, the method and/or kit is used for TR-F based monitoring of tropomyosin and/or actin interactions. In some embodiments, the method is for screening or identifying physiological conditions or compounds that affect cardiac myosin binding protein-C (cMyBP-C) binding to actin, actin filaments, and/or actin-tropomyosin complexes. In some embodiments, the method is a TR-F-based screen that detects changes in actin binding brought about by phosphorylation of the cMyBP-C N-terminal C0-C2 fragment.

In some embodiments, the methods described herein are for high throughput drug discovery or screening assay to identify drugs that mimic phosphorylation by inducing similar changes in binding that reduce actin-cMyBP-C binding with increased phosphorylation or by mutations (that reduce/decrease binding), or that enhance actin-cMyBP-C binding with decreased phosphorylation or by mutations (that enhance/increase binding) and measuring lifetime fluorescence change of phosphorylated-induced protein binding.

In some embodiments, the methods described herein are for mimicking phosphorylated state of cMyBP-C. In other embodiments, the method described herein are for screening drugs that modulate cMyBP-C (in either the phosphorylated or non-phosphorylated state) binding to actin-Tm. In further embodiments, the methods described herein are a complementary binding assay to be used in conjunction with cosedimentation.

In preferred embodiments, the labelling of a protein with a fluorescent probe is performed by industry standard technology.

### EXAMPLE

The following are non-limiting examples of the present invention. It is to be understood that said example is not intended to limit the present invention in any way. Equivalents or substitutes are within the scope of the present invention.

### Materials and Methods

Actin filament preparations. Actin was prepared from rabbit skeletal muscle by extracting acetone powder in cold water. The day prior to actin binding experiments (cosedimentation or TR-F), G-actin was polymerized by the addition of MgCl2 to a final concentration of 3 mM for 1 hour at 23° C. F-actin was collected by centrifugation at 4° C., 100,000 RPM (350,000×g) in a Beckman TLA-120.2 rotor and the pellet was resuspended in MOPS-actin binding buffer, M-ABB (100 mM KCl, 10 mM MOPS pH 6.8, 2 mM MgCl2, 0.2 mM CaCl₂), 0.2 mM ATP, 1 mM DTT, and 1 mM sodium azide). Any bundled actin was removed by centrifugation at 4° C., 15,000 RPM (21,000×g) for 10 min in an Eppendorf 5424R table-top microfuge. The resulting F-actin at approximately 30 μM was stabilized by the addition of an equimolar amount of phalloidin. After 10 min at room temperature, unbound phalloidin was removed by centrifugation at 4° C., 15,000 RPM (21,000×g) for 10 min in an Eppendorf 5424R table-top microfuge. F-actin was adjusted to 10 μM with MOPS-ABB. For actin-tropomyosin (Tm) binding, Tm was added to a ratio of 1:3.5 (Tm:actin) and allowed to incubate overnight.

Actin labeling. For fluorescence experiments (TR-F), actin was labeled at Cys-374 with 5-((((2-lodoacetyl)amino)ethyl)amino)Naphthalene-1-Sulfonic Acid (IAEDANS; Thermo Fisher Scientific, Waltham, MA). Labeling was done on F-actin. To 50 μM of G-actin in G-buffer (10 mM Tris pH 7.5, 0.2 mM CaCl2), 0.2 mM ATP), Tris pH 7.5 was added to a final concentration of 20 mM and then polymerized by the addition of 3 M KCl (to a final concentration of 100 mM) and 0.5 M MgCl2 (to a final concentration of 2 mM), followed by incubation at 23° C. for 1 hour. IAEDANS was added to a final concentration of 1 mM (from a 20 mM stock in DMF). Labeling was done for 3 hours at 23° C. and then overnight at 4° C. Labeling of actin with 2-(4′-(iodoacetamido)aniline)naphthalene-6-sulfonic acid (IAANS), N-((2-(iodoacetoxy)ethyl)-N-methyl)amino-7-nitrobenz-2-oxa-1,3-diazole (IANBD), and 7-Diethylamino-3-(4′-Maleimidylphenyl)-4-Methylcoumarin (CPM) were done the same as IAEDANS except with 250 μM IAANS and IANBD and 500 μM CPM for 1 h at 23° C. Labeling of actin with fluorescein-5-maleimide (FMAL) was done as for IAEDANS except labeling was for 5 h at 23° C. Labeling was terminated by the addition of DTT (to a final concentration of 5 mM). Labeled F-actin was collected by centrifugation for 30 min at 4° C., 100,000 RPM (350,000×g) in a Beckman TLA-120.2 rotor. The pellet was rinsed 3 times and then resuspended in labeling G-buffer (5 mM Tris pH 7.5, 0.2 mM CaCl2), and 0.5 mM ATP). G-actin was clarified by centrifugation for 10 min, 4° C., 90,000 RPM (290,000×g) in a Beckman TLA-120.2 rotor. G-actin was then re-polymerized by the addition of MgCl2 to 3 mM and incubation at 23° C. for 1 hour. Labeled F-actin was collected by centrifugation for 30 min at 4° C., 100,000 RPM (350,000×g) in a Beckman TLA-120.2 rotor. The pellet was washed 3 times and then resuspended in M-ABB. Bundled actin was removed and the labeled F-actin was stabilized with phalloidin and then complexed with Tm as described for unlabeled actin. Labeling efficiency was determined by measuring dye absorbance and protein concentration, measured with a BCA Protein Assay Kit (Thermo Fisher Scientific, Waltham, MA) using unmodified actin as a standard. The extent of labeling (dye/mol actin) was approximately 1.0 (IAEDANS), 1.0 (IAANS), 0.65 (CPM), and 0.3 (FMAL).

Recombinant human tropomyosin and tropomyosin-actin filament preparations. The gene encoding human α-Tm was inserted into a pET3d vector. Tm was expressed with 2 additional N-terminal amino acids (Ala-Ser) needed to ensure proper actin-binding and polymerization function as bacterially-expressed Tm is not otherwise functional due to lack of acetylation of the starting Met. Protein production in E. coli BL21(DE3)-competent cells (New England Biolabs, Ipswich, MA) was done in ZYP broth (1% tryptone, 0.5% yeast extract, 0.5% glycerol, 0.05% glucose, and 0.2% lactose) and pelleted by centrifugation in a Beckman JA-10 rotor at 1,800×g for 20 min at 4° C. Pellets were resuspended in ddH2O, brought to 1 M saline with solid NaCl, and homogenized using the Emulsiflex C3 (Avestin, Ontario, Canada). Cell debris of homogenized pellets were then pelleted by centrifugation in a Beckman JA-17 rotor at 28,950×g for 10 min at 4° C. and the supernatant containing expressed Tm was decanted into a glass beaker. The supernatant was boiled for 6 min and allowed to rest for 45 min at room temperature. The denatured lysate containing Tm was purified from other bacterial proteins by 3 total cycles of acid/base cuts (or until an A260/A280 ratio of <0.8 was reached): (i) precipitation of Tm by addition of 1 M HCl to pH ˜4.5, (ii) centrifugation at 28,950×g for 10 min at 4° C., (iii) resuspension of the pellets in 1 M KCl with addition of 1 M KOH to pH ˜7, (iv) centrifugation of the dissolved Tm at 28,950×g for 10 min at 4° C., and (v) collection of the supernatant. ˜1 g of α-Tm dimer was purified from 2 L of culture. Aliquots were stored at −80° C. until use. For preparation of fluorescent Tm-actin, Tm (in 1 M KCl) was diluted 10-fold with M-ABB lacking KCl (bringing the final concentration of KCl to 0.1 M) and reconstituted overnight in a 1:3.5, 1:5, or 1:7 molar ratio of Tm:actin. In preliminary experiments cosedimentation of actin and Tm was tested in 7:1 and 3.5:1 mixtures. As expected for 7:1 actin:Tm, almost all of the Tm was bound to actin and observed in the F-actin pellet and very low levels of Tm was in the supernatant. At the 3.5:1 actin:Tm ratio approximately 50% of the Tm pelleted with F-actin and 50% remained unbound in the supernatant.

Recombinant human cMyBP-C fragment preparations. pET45b vectors encoding E. coli optimized codons for the C0-C1 or C0-C2 portion of human cMyBP-C with N-terminal 6×His tag and TEV protease cleavage site were obtained from GenScript (Piscataway, NJ). In addition, C0-C2 mutants were generated with HCM mutations (R282W, E334K, L352P) or mutations in a positively-charged loop (RASK, residues 215-218) in the C1 domain that interacts with Tm. These Tm binding mutations introduce additional positive charges (A216R/S217K) or reverse charges (R215E/K218E). Mutations were engineered in the human cMyBP-C fragments using a Q5 Site-Directed Mutagenesis Kit (New England BioLabs, Ipswich, MA). All sequences were confirmed by DNA sequencing (Eton Biosciences, San Diego, CA). Protein production in E. coli BL21DE3-competent cells (New England Bio Labs, Ipswich, MA) and purification of C0-C1 and C0-C2 fusion proteins using His60 Ni Superflow resin was performed. cMyBP-C (His-tag removed by TEV protease digestion) was then concentrated, dialyzed to 50/50 buffer (50 mM NaCl and 50 mM Tris, pH 7.5) and stored at 4° C. Initial characterization of conditions for TR-F was done with this protein which was approximately 75% the correct molecular weight and 25% breakdown products (due to proteolysis within the motif between C1 and C2). For all of the TR-F binding curves and cosedimentation experiments the C0-C2 was further purified using size exclusion chromatography to achieve >90% intact C0-C2. For size exclusion chromatography C0-C2 (5-20 mg/ml, 1.7-2.5 ml) was applied in running buffer (150 mM NaCl, 50 mM NaPO4, 1 mM DTT, pH 6.7) to a HiPrep Sephacryl S-100 column. Flow rate was 0.7 ml/min. Purified C0-C2 was checked by SDS-PAGE for purity and then dialyzed into the appropriate buffer, usually M-ABB. Proteins were typically used for experiments within two weeks. For longer storage periods C0-C2 and C0-C1 proteins were stored at −20° C. in 50/50 buffer containing 50% glycerol, 1 mM DTT, protease inhibitors (Pierce 88265, 1 tablet/50 ml) and 1 mM sodium azide.

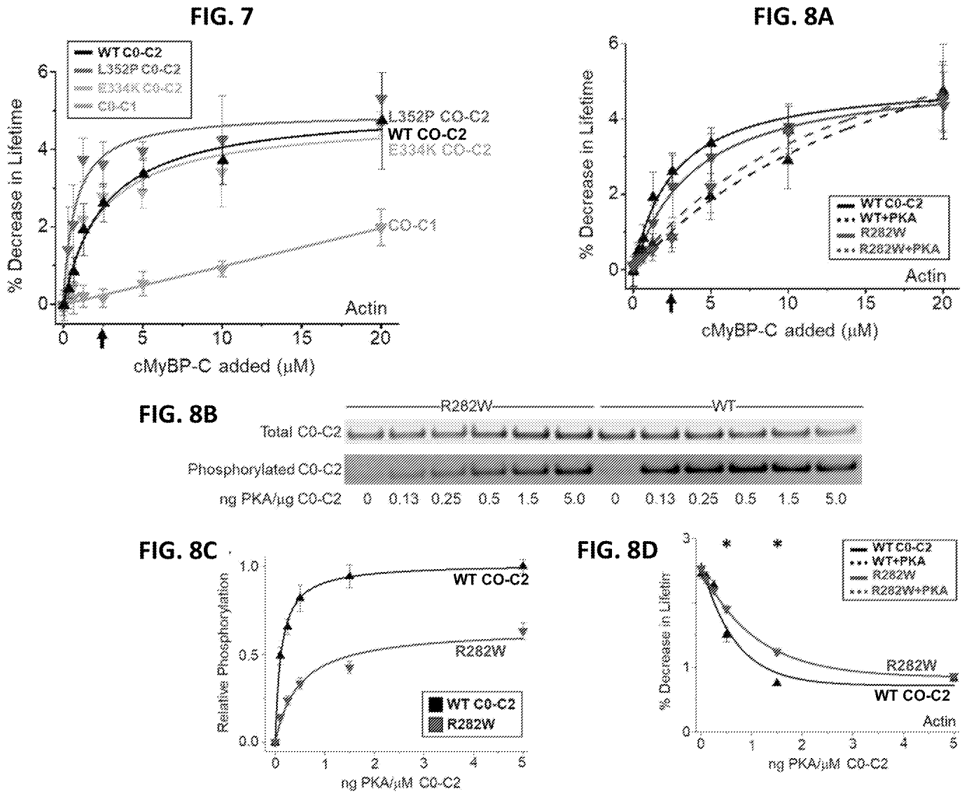

In vitro phosphorylation of cMyBP-C. For the determination of phosphorylated serines, phosphorylation was monitored by in-gel staining of proteins with Pro-Q Diamond (ThermoFischer, Waltham, MA) and staining total protein with SYPRO Ruby (ThermoFischer, Waltham, MA), according to the supplier's instructions. Maximal phosphorylation was shown to plateau at around 2.5 ng PKA/μg C0-C2 (FIG. 7A-7E). C0-C2 was typically treated with 7.5 ng PKA/μg C0-C2 unless otherwise noted. For liquid chromatography-tandem mass spectrometry (LC-MS/MS) C0-C2 WT and mutant R282W were treated with 65, 2.6, and 0.1 ng PKA/μg C0-C2. Phosphorylated proteins were separated by SDS-PAGE. The C0-C2 bands were excised. Following LC-MS/MS a probability-based approach was used for high-throughput protein phosphorylation analysis and site localization.

Actin cosedimentation assays. Actin binding by cMyBP-C fragments (C0-C2 or C0-C1) was determined by cosedimentation. Binding levels were determined at 25° C. in M-ABB using 1 μM F-actin or 1 μM F-actin containing 0.29 μM Tm incubated with increasing amounts of C0-C2 or C0-C1. All cosedimentation curves were generated from at least 2 separate purifications of cMyBP-C (and actin); n>4 (typically n=6) for all data points. All reaction mixtures were made separately and not prepared in a single batch.

Determination of Bₘₐₓ and K_{d} values. The maximum molar binding ratio (Bₘₐₓ) and dissociation constant (K_{d}) values for C0-C2 binding to actin were determined by fitting the data to a quadratic model (Michaelis-Menten function) using Origin Pro 2019 computer software package through a non-linear least-squares minimization (Levenberg Marquardt iteration algorithm). χ² values of quadratic fits for all binding experiments were <0.005. The exception to this is C0-C1 and the R215E/K218E mutants of C0-C2 when phosphorylated. In these cases, poor fits were due to much reduced, non-saturable, binding and they instead fit to a linear function. These apparent K_{d} and Bₘₐₓ values are used as comparative indicators of binding characteristics for C0-C2 binding to actin under different conditions (− and + phosphorylation or the presence of mutations). They represent the apparent dissociation constants (C0-C2 concentration required for half-maximal binding) and maximal binding ratios of the MyBP-C fragments to actin (C0-C2/actin) in co-sedimentation experiments where bound MyBP-C and total actin monomers are directly measured. In TR-F generated curves the Kd values again represent the apparent dissociation constants (C0-C2 concentration required for half-maximal binding) but the Bₘₐₓ in this case is the maximal change in lifetime of IAEDANS when C0-C2 binding is maximal. Kd values, expressed as μM C0-C2 in both assays, can be compared within and between the two assays (cosedimentation or TR-F). The Bₘₐₓ values, having different units in the two assays, can only be compared within the same type of assay (cosedimentation or TR-F). These values derived from fitting the data of the binding curves to a quadratic model (Michaelis-Menten function) are the standard values used to describe binding of MyBP-C fragments to filamentous actin.

While useful for comparing curves, it is questionable how relevant the numbers are to in vivo binding. Binding is likely to be complex under the conditions required for binding curves as each actin monomer in the F-actin possesses multiple interaction sites for C0-C2. Likewise, C0-C2 possesses multiple binding sites for multiple actin regions. At elevated C0-C2 concentrations, competition between sites, cooperative interactions, and steric hindrance are all likely to be significant. In vivo, high ratios of C0-C2:actin (greater than around 1:7) are not present and therefore neither competition nor steric hindrance is a factor. Visual inspection of each curve confirms that when lower K_{d} values are observed, more binding is observed at low (sub-stoichiometric) C0-C2:actin levels that are more physiologically relevant. Both K_{d} and Bₘₐₓ values are reported but the Kd values and visual inspection of the curves at low C0-C2 concentrations are the more physiologically relevant for comparison of binding.

TR-F data acquisition. 50 μl of sample aliquots were loaded manually with a multichannel pipette in 384-well black polypropylene microplates (#781209, Greiner Bio-One, Monroe, NC). Plates were spun 1 min at 1,000 RPM (200×g) in Eppendorf rotor 5810R A-4-81) to remove air bubbles. Fluorescence lifetime measurements were acquired using a high-precision fluorescence lifetime plate reader (FLTPR; Fluorescence Innovations, Inc., Minneapolis, MN). Dye-labeled F-actin (alone or mixed with unlabeled C0-C2) was excited with either a 355-nm (Teem Photonics, Meylan, France) for 1,5-IAEDANS, 5-((((2-lodoacetyl)amino)ethyl)amino)Naphthalene-1-Sulfonic Acid (IAEDANS), 2-[4′-(iodoacetamido)anilino]naphthalene-6-sulfonic acid (IAANS), and 7-Diethylamino-3-(4′-Maleimidylphenyl)-4-Methylcoumarin (CPM) dyes or a 473-nm microchip laser (Bright Solutions, Cura Carpignano, Italy) for Fluorescein-5-Maleimide (FMAL) and N-((2-(iodoacetoxy)ethyl)-N-methyl)amino-7-nitrobenz-2-oxa-1,3-diazole (IANBD) dyes. Emission was filtered with 409-nm long pass and 470/20-nm band-pass filter or 488-nm long pass and 517/20-nm band pass filters, respectively (Semrock, Rochester, NY). The FLTPR allows for high-throughput fluorescence lifetime detection at high precision by using unique direct waveform-recording technology.

TR-F analysis. TR-F waveforms for each well were fitted to a single-exponential decay using least-squares minimization global analysis software (Fluorescence Innovations, Inc.). The decay of the excited state of the fluorescent dye attached to actin at Cys374 to the ground state is:

$$I ( t ) = {I}_{0} exp ( - t / τ ) , \qquad ( Eq . 1 )$$

where I₀ is the fluorescence intensity upon excitation (t=0) and T is the fluorescence lifetime (t=τ when I decays to 1/e or ˜37% of I₀).

HTS data analysis. For suitability in high-throughput screening (HTS), TR-F assay quality was determined for various comparisons of sample conditions (i.e., unbound vs. bound actin and unphosphorylated vs. phosphorylated C0-C2). Typically, 40-60 wells of each condition were analyzed. To stimulate future drug screens, 1% DMSO was added to each sample mixture. Sample mixtures were IAEDANS-actin, IAEDANS-actin+C0-C2, IAEDANS-actin+phosphorylated C0-C2, IAEDANS-actin-Tm, IAEDANS-actin-Tm+C0-C2, and IAEDANS-actin-Tm+phosphorylated C0-C2. Comparison of pairs of samples (sample A versus sample B) was indexed by the Z′ factor where a value of 0 to 0.5 indicates good and 0.5 to 1.0 indicates excellent assay quality:

$${Z}^{′} = 1 - 3 ( \frac{{σ}_{A} + {σ}_{B}}{{μ}_{A} - {μ}_{B}} ) \qquad ( Eq . 2 )$$

where σ_{A} and σ_{B} are the standard deviations (SDs) of the controls τ_{A} and τ_{B}, respectively, and μ_{A} and μ_{B} are the means of the controls T_{A} and TB, respectively. Here, two sample comparisons A versus sample B are made: i) unbound IAEDANS-actin (sample A) versus C0-C2-bound IAEDANS-actin (sample B), and ii) C0-C2 (−PKA treatment)-bound IAEDANS-actin (sample A) versus C0-C2 (+PKA treatment)-6 bound IAEDANS-actin (sample B). The same two comparisons are also done with IAEDANS-actin-Tm.

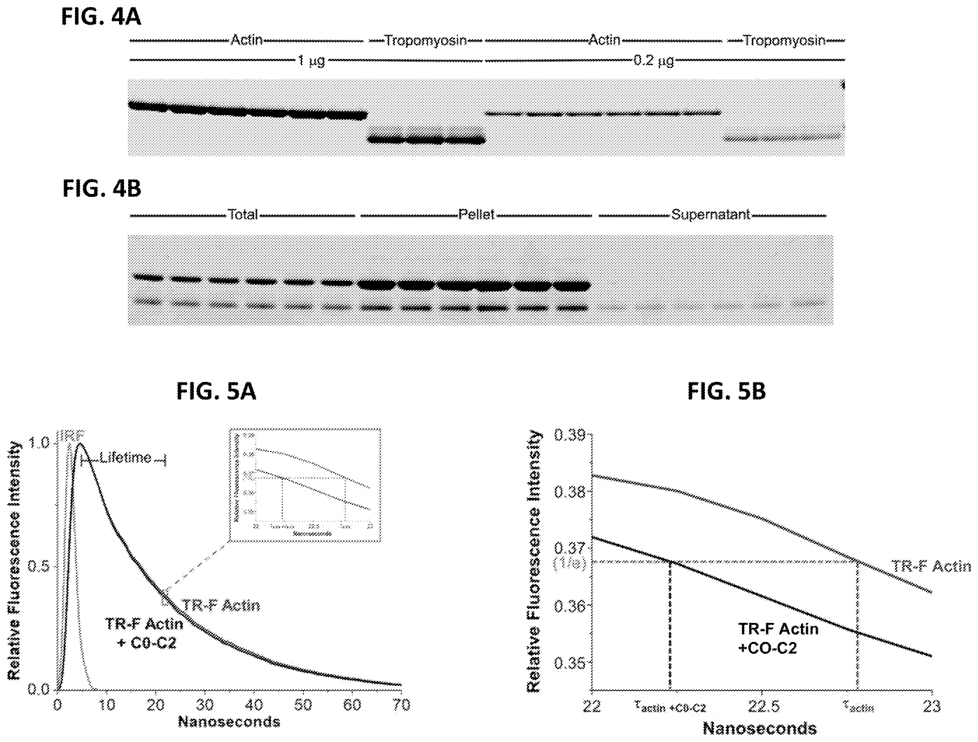

Statistics. Sample means are from four or more independent experiments. Each experiment, following the optimization of binding conditions, was carried out using at least two independent protein preparations. Average data are provided as mean±SE except for Table 3 and FIG. 4 which used±SD for Z′scores. Statistical significance is evaluated by use of an unpaired t-test. P values <0.05 were taken as indicating significant differences, as defined in the Figure and Table legends.

### Results

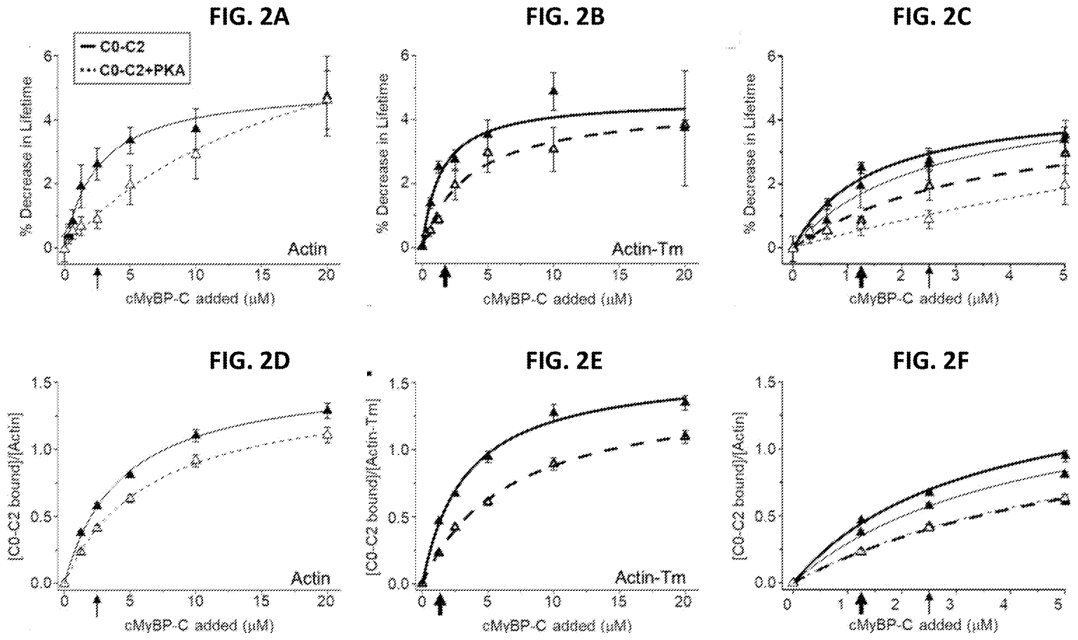

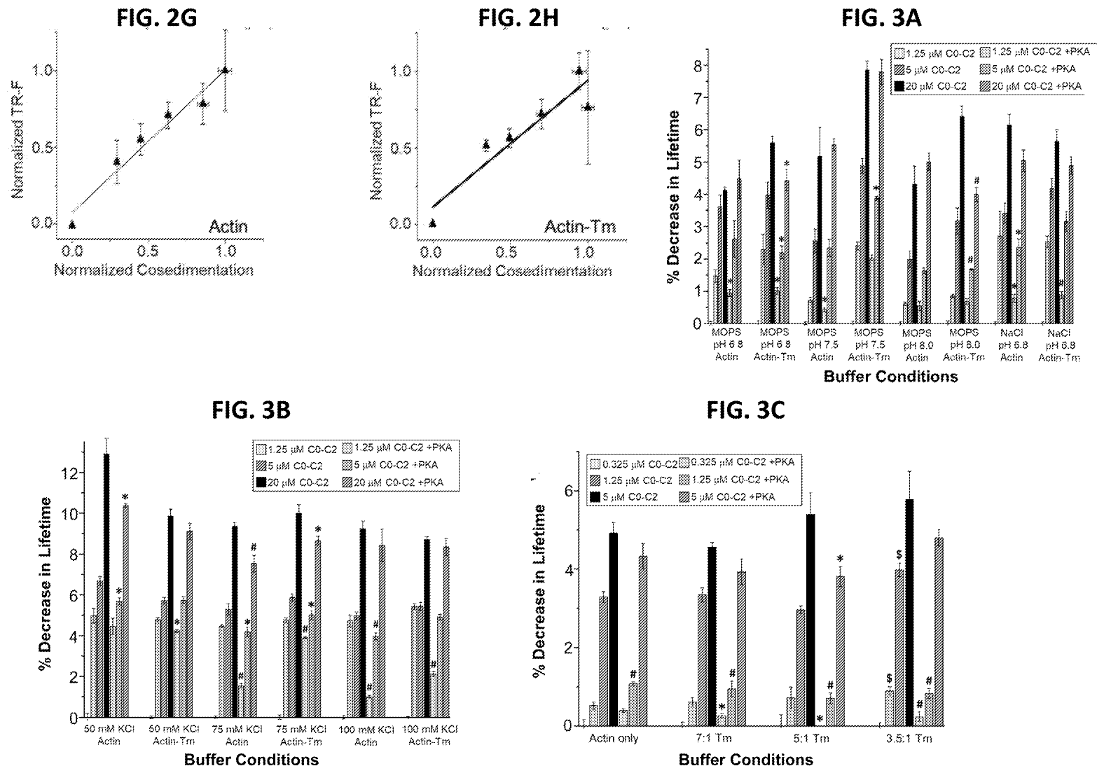

Testing five fluorescent probes for suitability in TR-F actin binding assays. To find a suitable fluorescent dye for the TR-F assay, actin was labeled on its surface-exposed cysteine (Cys-374) with 5 different thiol-reactive dyes. Decays of fluorescence from labeled actin and actin in the presence of 5 μM and 20 μM C0-C2 fragments were analyzed using Eq. 1 (Materials and Methods, above). Changes in lifetime due to binding of unphosphorylated and phosphorylated (by PKA) C0-C2 were compared (FIGS. 1A-1B) At both 1 μM and 5 μM actin (first bars (1) in FIGS. 1A and 1B) the reduction in lifetime due to the binding of 5 and 20 μM C0-C2 (bars 2 & 3) was highly significant (p<0.000002) for IAEDANS, IAANS, CPM, and FMAL. For IANBD, significant effects were observed at 1 μM and 5 μM labeled actin with 20 μM C0-C2 (p<0.000002 and p<0.015). 5 μM C0-C2 incubated with IANBD-actin (at 1 μM and 5 μM) showed a reduction in lifetime that trended to but did not reach significance (p=0.058 and 0.162). Phosphorylation, known to reduce C0-C2 binding to F-actin, significantly reduced the effect on the lifetime changes—it reduced the reduction in lifetime. Comparison of unphosphorylated versus phosphorylated C0-C2 (FIGS. 1A and 1B, (2) versus (4) and (3) versus (5)) showed significant changes (p<0.05) that were observed for all probes, though not at all actin and C0-C2 concentrations. For IAANS-, CPM- and FMAL-labeled actin, the differences due to phosphorylation were significant at both actin and C0-C2 concentrations. For IAEDANS-labeled actin, the differences due to phosphorylation were significant for 1 μM actin with 5 and 20 μM C0-C2 and for 5 μM actin with 5 μM C0-C2, but not 5 μM actin with 20 μM C0-C2 (p=0.24). Lack of significant differences due to phosphorylation at a high concentration (20 μM) of C0-C2 is not unexpected as phosphorylated C0-C2 “catches up” with non-phosphorylated C0-C2 at maximal binding (see binding curve in FIG. 2A). 1 μM IANBD-labeled actin and 20 μM C0-C2 did show a significant difference upon phosphorylation of C0-C2 (p=0.024). The other 3 conditions were tested using IANBD-actine (1 μM IANBD-actin with 5 μM C0-C2, 5 μM IANBD-actin with 5 or 20 μM C0-C2) showed no significant difference

Actin-cMyBP-C TR-F biosensor. A time-resolved fluorescence (TR-F) based screen was developed to detect changes in actin binding brought about by different experimental conditions. A TR-F actin binding assay in a 384-well plate format made possible the testing of variables such as pH, ionic strength, phosphorylation of cMyBP-C, and the presence of mutations in cMyBP-C. The results indicate that the assay will be useful in identifying therapeutic drugs that can modify cMyBP-C actin binding properties to the same extent as phosphorylation. This same approach should be suitable, with appropriate modifications to the study of other actin binding proteins (ABPs).

Using the aforementioned TR-F binding assay, the lifetime of the thiol reactive dye IAEDANS that is covalently attached to F-actin's readily labeled cysteine (Cys-374) was monitored. The buffer for actin binding experiments was MOPS-Actin Binding Buffer (M-ABB) (100 mM KCl, 10 mM MOPS pH 6.8, 2 mM MgCl2, 0.2 mM CaCl2), 0.2 mM ATP, 1 mM DTT, and 1 mM sodium azide). These conditions allowed for distinguishing between binding of C0-C2 that was unphosphorylated or phosphorylated (by PKA) (FIG. 2A) to actin or actin-Tm (FIG. 2B). The addition of C0-C2 decreased the lifetime of IAEDANS-actin (with and without Tm) in a concentration-dependent manner. Upon phosphorylation of C0-C2 with PKA the decrease in IAEDANS-actin lifetime was much less, at submaximal binding concentrations of C0-C2 (FIG. 2A-2H and Table 1). This indicates that PKA treatment reduced cMyBP-C binding to actin and actin-Tm (FIG. 2A-2B, dashed versus solid lines). These conditions also showed the presence of Tm on actin increases C0-C2 binding (FIG. 2C, thick versus thin lines). At substoichiometric levels of binding (1.25 μM C0-C2) Tm increased binding by 41% (p<0.005) (Table 2). These data were compared to results derived from cosedimentation experiments done under the same conditions (FIG. 2D-2F). Over the same concentration range, TR-F and cosedimentation assays provided similar binding profiles for unphosphorylated C0-C2, and these were linearly correlated (For actin and unphosphorylated C0-C2, R2=0.96, adjusted-R2=0.95; FIG. 1G. For actin-Tm R2=0.87, adjusted-R2=0.84, FIG. 2H). However, subtle differences in K_{d} and Bₘₐₓ are observed when fit to a quadratic equation for Michaelis-Menten binding kinetics (Table 1 or FIG. 2G). Cosedimentation showed a less dramatic effect of PKA on binding actin compared with the TR-F assay (FIG. 2D versus FIG. 2A, Table 1) while the effects were similar on actin-Tm (FIG. 2E versus FIG. 2B, Table 1). The effect of PKA phosphorylation of C0-C2 is optimally seen when C0-C2 levels are below saturation (substoichiometric) at 2.5 μM C0-C2 for actin (thin arrows in FIGS. 2A, 2C, 2D, and 2F) and 1.25 μM C0-C2 for actin-Tm (thick arrows in FIGS. 2B, 2C, 2E and 2F). This is the case for both TR-F and cosedimentation assays. For the TR-F assay, these concentrations showed the largest absolute difference in the change in lifetime between minus and plus PKA, of 1.7% and 2.0% (Table 2). These equate to PKA treatment of C0-C2 reducing the change in lifetime of IAEDANS on actin by 66% and 74% compared to the change in lifetime observed when C0-C2 was not phosphorylated (Table 2). These concentrations (2.5 μM C0-C2 for actin, and 1.25 μM C0-C2 for actin-Tm) also result in the highest levels of statistical significance when comparing minus and plus PKA, p=2.9×10⁻¹¹ for actin and p=1.6×10⁻¹³ for actin-Tm.

TABLE 1
TR-F and cosedimentation binding parameters
of cMyBP-C binding to actin and actin-Tm.

| | | | | | | Bₘₐₓ |
|---|---|---|---|---|---|---|
| | | | | | | (Fraction bound; |
| Construct | Filament | PKA | K_{d} (μM) | Bₘₐₓ (Δτ) | K_{d} (μM) | C0-C2/actin) |
| C0-C2 | Actin | − | 2.5 ± 0.4 | 5.1 ± 0.3 | 4.3 ± 0.3 | 1.56 ± 0.04 |
| C0-C2 | Actin | + | 19 ± 5^{#} | 9 ± 1^{#} | 6.5 ± 0.3^{#} | 1.48 ± 0.03 |
| C0-C2 | Actin-Tm | − | 1.4 ± 0.5^{$} | 4.6 ± 0.4 | 3.3 ± 0.4 | 1.61 ± 0.06 |
| C0-C2 | Actin-Tm | + | 3.6 ± 0.7^{#}* | 4.5 ± 0.3* | 6.6 ± 0.4^{#} | 1.46 ± 0.04 |

In Table 1, data are mean±S.E. (n>4) for a binding curve of 8 concentrations of C0-C2 (0-20 μM) to 1 μM of actin (FIG. 2A-2H) fit to a quadratic equation. Significant difference due to PKA treatment (− vs. +PKA rows), ^{#}p<0.05. Significant difference due to presence of Tm (Actin vs. Actin-Tm rows of same PKA condition), *p<0.05. Trending differences in Kd due to the presence of Tm, ^{$}p=0.10. Units for Bₘₐₓ are lifetime change (maximal Δτ) for TR-F and fraction of actin bound (C0-C2/actin) for cosedimentation. See methods for a detailed discussion of K_{d} and Bₘₐₓ uses and limitations.

TABLE 2
Change in IAEDANS Lifetime for IAEDANS-
Actin and IAEDANS-Actin-Tm upon substoichiometric
binding of C0-C2 and C0-C1.

| | | % Change in | |
|---|---|---|---|
| cMyBP-C complex with 1 μM F-actin | | IAEDANS Lifetime | |
| Construct | IAEDANS- | −PKA | +PKA |
| C0-C2 Wild type | Actin | 2.61 ± 0.14 | 0.88 ± 0.08 |
| (2.5 μM) | | | |
| C0-C2 Wild type | Actin | 1.92 ± 0.17 | 0.68 ± 0.08 |
| (1.25 μM) | | | |
| C0-C2 Wild type | Actin | 0.83 ± 0.1 | |
| (0.625 μM) | | | |
| C0-C2 Wild type | Actin-Tm | 2.70 ± 0.07 | 0.71 ± 0.06 |
| (1.25 μM) | | | |
| C0-C2 Wild type | Actin-Tm | 1.70 ± 0.12 | |
| (0.65 μM) | | | |
| C0-C2 Wild type | Actin-Tm | 0.56 ± 0.06 | |
| (0.0313 μM) | | | |
| C0-C1 (Δm-C2) | Actin | 0.17 ± 0.06 | |
| (2.5 μM) | | | |
| C0-C2 L352P | Actin | 3.62 ± 0.19 | |
| (2.5 μM) | | | |
| C0-C2 E334K | Actin | 2.79 ± 0.11 | |
| (2.5 μM) | | | |
| C0-C2 R282W | Actin | 2.20 ± 0.24 | 0.86 ± 0.12 |
| (2.5 μM) | | | |
| C0-C2 R282W | Actin | 1.24 ± 0.12 | 0.53 ± 0.08 |
| (1.25 μM) | | | |
| C0-C2 R215E/K218E | Actin | 0.77 ± 0.06 | |
| (1.25 μM) | | | |
| C0-C2 R215E/K218E | Actin-Tm | 0.48 ± 0.05 | |
| (1.25 μM) | | | |
| C0-C2 A216R/S217K | Actin | 2.63 ± 0.10 | |
| (1.25 μM) | | | |
| C0-C2 A216R/S217K | Actin | 2.16 ± 0.13 | |
| (0.625 μM) | | | |
| C0-C2 A216R/S217K | Actin-Tm | 2.98 ± 0.08 | |
| (1.25 μM) | | | |
| C0-C2 A216R/S217K | Actin-Tm | 2.68 ± 0.05 | |
| (0.625 μM) | | | |
| C0-C2 A216R/S217K | Actin-Tm | 2.08 ± 0.12 | |
| (0.313 μM) | | | |

Table 2 shows substoichiometric binding levels of cMyBP-C C0-C2 and C0-C1 on 1 μM IAEDANS-labeled actin and actin-Tm. Data is provided as mean±SE (n>4). P values comparing different samples are described herein.

Optimizing TR-F assay conditions. Using 1 μM IAEDANS labeled F-actin, different pH, ionic conditions, and Tm levels were tested to determine the optimal values for use in subsequent experiments (FIG. 3A-3C).

Increasing pH increases the difference between actin and actin-Tm binding to C0-C2. Varying the pH of MOPS-Actin Binding Buffer (M-ABB), between 6.8 and 8.0 subtly altered TR-F lifetime changes reported for binding to bare actin or actin decorated with tropomyosin (Tm) (FIG. 3A). Tm itself did not significantly change the lifetime of the_IAEDANS labeled F-actin. Both lifetimes were around 17.60 nanoseconds (see actin alone and actin-Tm alone values in Table 2. In the presence of Tm, at pH 6.8, changes in TR-F due to unphosphorylated C0-C2 binding were greater than on bare actin (p=0.16, 0.53, and 0.001 for 1.25, 5 and 20 μM C0-C2). At higher pH (7.5 and 8.0), the differences between actin and actin-Tm upon binding to C0-C2 were more significant (p<0.04 for all unphosphorylated C0-C2 concentrations).

NaCl reduces Tm effects. At pH 6.8, replacing the 100 mM KCl (in M-ABB) for 100 mM NaCl decreases the effect of Tm on binding of C0-C2 when unphosphorylated ((p=0.83, 0.12, and 0.33 for 1.25, 5 and 20 μM C0-C2) (FIG. 3A).

Increasing pH reduces effects of C0-C2 PKA phosphorylation on TR-R measured binding. TR-F detection of binding to bare actin and actin-Tm was responsive to PKA phosphorylation of C0-C2 at the lower C0-C2 concentrations. The responsiveness was reduced as the pH was increased from 6.8 to 7.5 and 8.0 (FIG. 3A). At pH 6.8 with 100 mM KCl, for actin plus 1.25 and 5 μM C0-C2 and actin-Tm plus 1.25 and 5 μM C0-C2, these differences were typically significant when comparing minus and plus PKA. (p=0.036, 0.173, 0.01 and 0.02, respectively; average p=0.060). Increasing the pH to 7.5 or 8.0 reduced the responsiveness to phosphorylation. At pH 7.5, the same comparisons of minus and plus PKA gave p=0.015, 0.649, 0.053 and 0.003; average p=0.180. At pH 8.0, the same comparisons of minus and plus PKA gave p=0.649, 0.224, 0.103 and 0.004; average 0.245.

NaCl increases PKA phosphorylation effects. Replacing KCl with NaCl, increased changes due to PKA treatment. At pH 6.8 with 100 mM NaCl, for actin plus 1.25 and 5 μM C0-C2 and actin-Tm plus 1.25 and 5 μM C0-C2, differences were all significant when comparing minus and plus PKA (p=0.039, 0.030, 0.00003 and 0.06; average p=0.032).

100 mM KCl increases detection of PKA phosphorylation. M-ABB at pH 6.8 with KCl was further tested for the effects of different KCl concentrations. A greater effect of phosphorylation on TR-F at 100 mM KCl was observed than at 50 or 25 mM KCl. At 1.25 μM and 5 μM C0-C2 (levels showing submaximal binding) the 100 mM KCl level resulted in the largest differences between phosphorylated and unphosphorylated C0-C2 binding (FIG. 3B). These largest differences were observed at 1.25 μM C0-C2, where the lifetime change was reduced, upon phosphorylation, by 79% for actin and 61% for actin-Tm (compare bar (1) with bar (4) in FIG. 3B). This difference is due mainly to a reduction in the binding of phosphorylated C0-C2 as the KCl concentration was increased.

Actin-Tm ratio of 3:5:1 displays significant Tm and PKA phosphorylation effects on TR-F lifetime changes. At pH 6.8 and 100 mM KCl we compared the effect of different ratios of actin to Tm on the ability of TR-F to detect changes in the lifetime of IAEDANS-actin mediated by PKA phosphorylation of C0-C2 (FIG. 3C). At 7:1 (actin:Tm), binding of C0-C2 was not significantly different than that seen with actin alone. Comparison of changes in lifetime between actin and 7:1 (actin:Tm) at 3 concentrations (0.325, 1.25 and 5 μM) of unphosphorylated C0-C2 gave average p values of 0.54, 0.85 and 0.27. When Tm was included at higher concentrations (5:1 and 3.5:1 actin:Tm), increases in the lifetime changes were observed. The p values for 5:1 actin:Tm compared with actin alone over the 3 concentrations of C0-C2 were 0.53, 0.09 and 0.44. For 3.5:1, the p values were 0.03, 0.03 and 0.29 as the lifetimes were reduced compared to those observed with actin alone. Therefore, increasing Tm to 5:1 or 3.5:1 increased the effects of C0-C2 on lifetime changes, presumably by increasing binding. At 5:1 and 3.5:1 actin:Tm, the greatest effects (changes in lifetimes) due to PKA phosphorylation were observed at 1.25 μM C0-C2 (bar (2) versus bar (5), FIG. 3C). At this concentration of C0-C2, the change in lifetime due to PKA phosphorylation was reduced 76% (from a lifetime change of 2.96% to 0.71%) at 5:1 actin:Tm and at 3.5:1, the reduction was 79% (from a lifetime change of 3.98% to 0.83%). To maintain the maximal effect of Tm, the 3.5:1 (actin:Tm) ratio was used in the subsequent experiments. Cosedimentation experiments confirmed that at this ratio, 3.5:1, Tm was in excess of the amount needed (7:1, actin:Tm) to decorate F-actin. Approximately ½ of the Tm cosedimented with the F-actin and % remained in solution (FIG. 4A-4B).

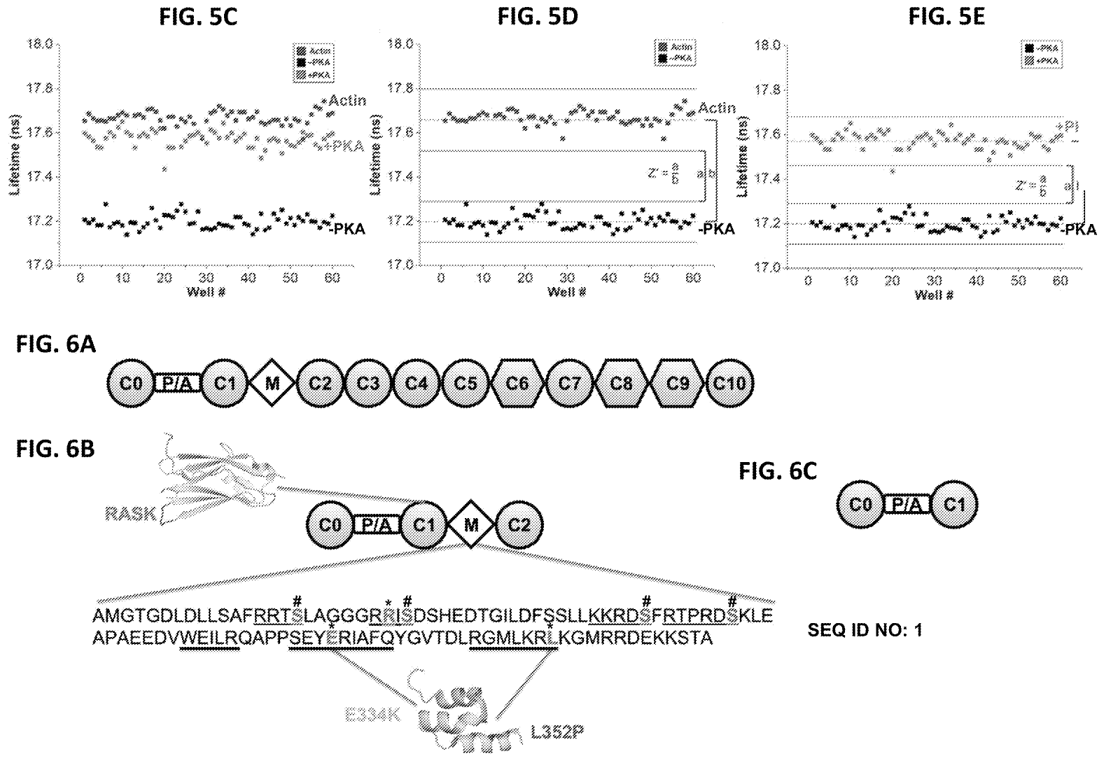

Z′ score evaluation of TR-F binding assays for HTS. Z′ scores were used to determine if actin-cMyBP-C TR-F sensor has sufficient sensitivity for employment in high-throughput screens (HTS) to identify drugs that effect binding of cMyBP-C or the mimic the effects of phosphorylation of cMyBP-C on actin binding. Z′ scores (see Eq. 2 in Materials and Methods) allow the determination of whether the signal window (Δτ) between two states (bound versus not bound or phosphorylated binding versus not phosphorylated binding) and the variance (SD) or the two states indicate a worthless HTS (Z′score <0), a doable HTS (Z′ score 0-0.5) or an excellent HTS assay quality (Z′ score 0.5-1.0) (see also FIG. 5D-5E). Using either the IAEDANS-actin or IAEDANS-actin-Tm sensor, a simulated HTS was performed by loading ˜60 wells of each condition in 384-well plates. The conditions tested were: actin alone, actin plus C0-C2, and actin plus PKA-treated C0-C2 as well as actin-Tm alone, actin-Tm plus C0-C2, and actin-Tm plus PKA-treated C0-C2. Means and 3× standard deviations of the lifetimes for each condition were used to determine the Z′ scores (FIG. 5A-5E). The Z′ factor for the screen shown in FIG. 5A-5E was calculated as Z′=0.56 for IAEDANS-actin alone versus IAEDANS-actin plus C0-C2 and Z′=0.27 for IAEDANS-actin plus C0-C2 versus IAEDANS-actin plus PKA-treated C0-C2. The Z′ score determination was performed in triplicate with three different IAEDANS-actin (and IAEDANS-actin-Tm) and C0-C2 preparations. All tests gave Z′ scores that indicated good (doable) or excellent screening quality with actin-Tm showing higher scores than actin-alone (Table 3). Actin-Tm showed higher scores (average Z′=0.46) than actin alone (average Z′=0.29) (Table 3). These results validate the robustness of TR-F for HTS screening

TABLE 3
Z′ score quality calculation for
potential actin C0-C2 binding screens.

| | | # of | Lifetime | | Z′ |
|---|---|---|---|---|---|
| (#) Preparation | PKA | wells | (ns) | 3× SD (ns) | score |
| (1) Actin alone | − | n = 48 | 17.53 | 0.08 | |
| | | | | Actin ± C0-C2 | 0.43 |
| (1) Actin + C0-C2 | − | n = 58 | 16.97 | 0.24 | |
| | | | | Actin | 0.07 |
| | | | | C0-C2 ± PKA | |
| (1) Actin + C0-C2 | + | n = 60 | 17.34 | 0.10 | |
| (2) Actin alone | − | n = 57 | 17.68 | 0.08 | |
| | | | | Actin ± C0-C2 | 0.56 |
| (2) Actin + C0-C2 | − | n = 60 | 17.22 | 0.13 | |
| | | | | Actin | 0.27 |
| | | | | C0-C2 ± PKA | |
| (2) Actin + C0-C2 | + | n = 59 | 17.54 | 0.11 | |
| (3) Actin alone | − | n = 10 | 17.59 | 0.25 | |
| | | | | Actin ± C0-C2 | 0.12 |
| (3) Actin + C0-C2 | − | n = 49 | 17.06 | 0.21 | |
| | | | | Actin | 0.26 |
| | | | | C0-C2 ± PKA | |
| (3) Actin + C0-C2 | + | n = 48 | 17.49 | 0.10 | |
| (1) Actin-Tm alone | − | n = 60 | 17.52 | 0.08 | |
| | | | | Actin-Tm ± | 0.51 |
| | | | | C0-C2 | |

| | | | | |
|---|---|---|---|---|
| (1) Actin-Tm + C0-C2 − | n = 60 | 16.91 | 0.21 | |
| | | | Actin-Tm | 0.26 |
| | | | C0-C2 ± PKA | |
| (1) Actin-Tm + C0-C2 + | n = 58 | 17.39 | 0.09 | |
| (2) Actin-Tm alone − | n = 48 | 17.66 | 0.14 | |
| | | | Actin-Tm ± | 0.50 |
| | | | C0-C2 | |
| (2) Actin-Tm + C0-C2 − | n = 58 | 17.20 | 0.09 | |
| | | | Actin-Tm | 0.45 |
| | | | C0-C2 ± PKA | |
| (2) Actin-Tm + C0-C2 + | n = 60 | 17.57 | 0.11 | |
| (3) Actin-Tm alone − | n = 10 | 17.60 | 0.08 | |
| | | | Actin-Tm ± | 0.57 |
| | | | C0-C2 | |
| (3) Actin-Tm + C0-C2 − | n = 48 | 17.09 | 0.14 | |
| | | | Actin-Tm | 0.45 |
| | | | C0-C2 ± PKA | |
| (3) Actin-Tm + C0-C2 + | n = 49 | 17.51 | 0.09 | |

Three separate preparations of actin and C0-C2 were used (#: 1, 2, and 3) to run mock plate reader screens for calculating Z′ score using Eq. 2 (see Materials and Methods, above) based on mean lifetime (ns) and standard deviation (SD). For binding experiments using IAEDANS-actin, 2.5 μM of C0-C2 was added. 1.25 μM of C0-C2 was used for IAEDANS-actin-Tm. Binding was done with (+) or without (−) PKA treatment to phosphorylate cMyBP-C. Within each preparation, actin alone was compared to actin with bound C0-C2 for one comparison (Actin±C0-C2; first row vs. second row for each preparation). Actin bound to unphosphorylated vs. phosphorylated C0-C2 was also compared (Actin C0-C2±PKA; second row vs. third row for each preparation). See FIG. 5C-5E, for a representative example of the mock screen

Effects of cMyBP-C HCM and Tm-binding mutations on actin binding detected by TR-F. The ability of the TR-F binding assay to quickly and reproducibly distinguish between two states of cMyBP-C suggests that it will be useful in probing the effects of mutations in cMyBP-C (as well as other ABPs) on actin binding. Importantly, the ability to detect effects of mutations of actin binding would serve as proof of principle that the assay can detect other factors, such as therapeutic drugs, that modulate binding. For these reasons the sensitivity of the TR-F assays to detect changes in the C0-C2 binding due to 3 hypertrophic cardiomyopathy (HCM) mutations predicted to affect actin binding in very different manners. The L352 mutation increases actin binding whereas the E334K decreases actin binding. The R282W mutation is predicted to alter phosphorylation and would thereby alter actin binding. Along with the 3 HCM mutations, 2 mutations in a proposed Tm binding sequence in C1 were also tested and are diagrammed in FIG. 6A-6C. The key comparisons that validate the assay's usefulness to detect a variety of changes in binding due to mutations are presented in FIG. 7-9D and Table 2. As the assay makes possible the comparison of combinations of multiple factors (minus and plus HCM or Tm-binding mutations, minus and plus phosphorylation by PKA minus and plus Tm) this was done as well.

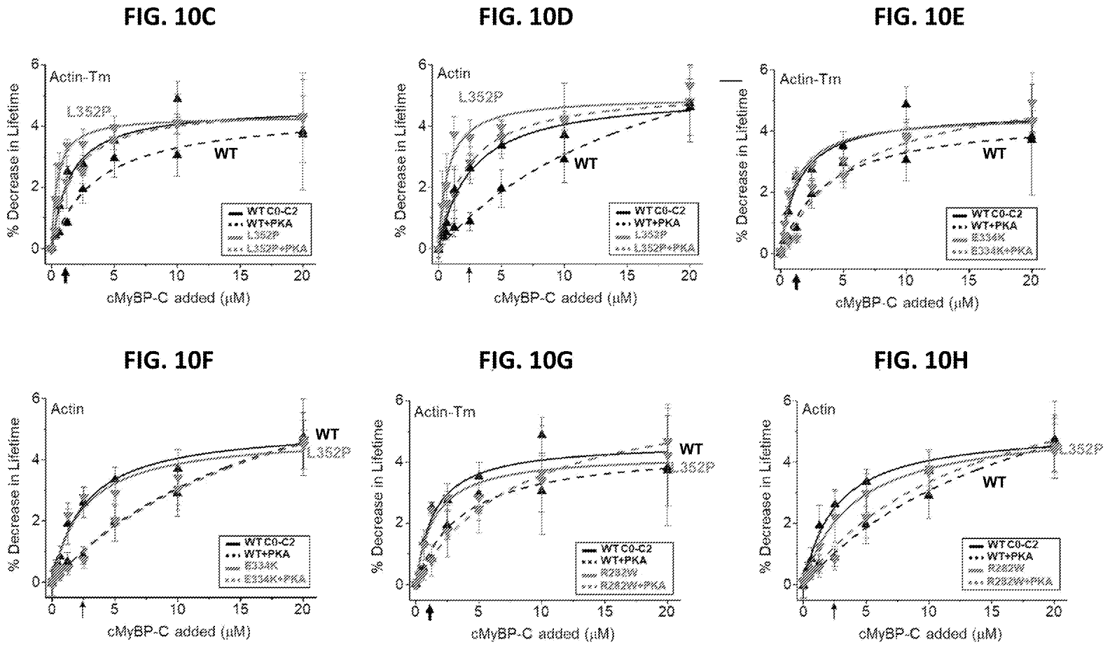

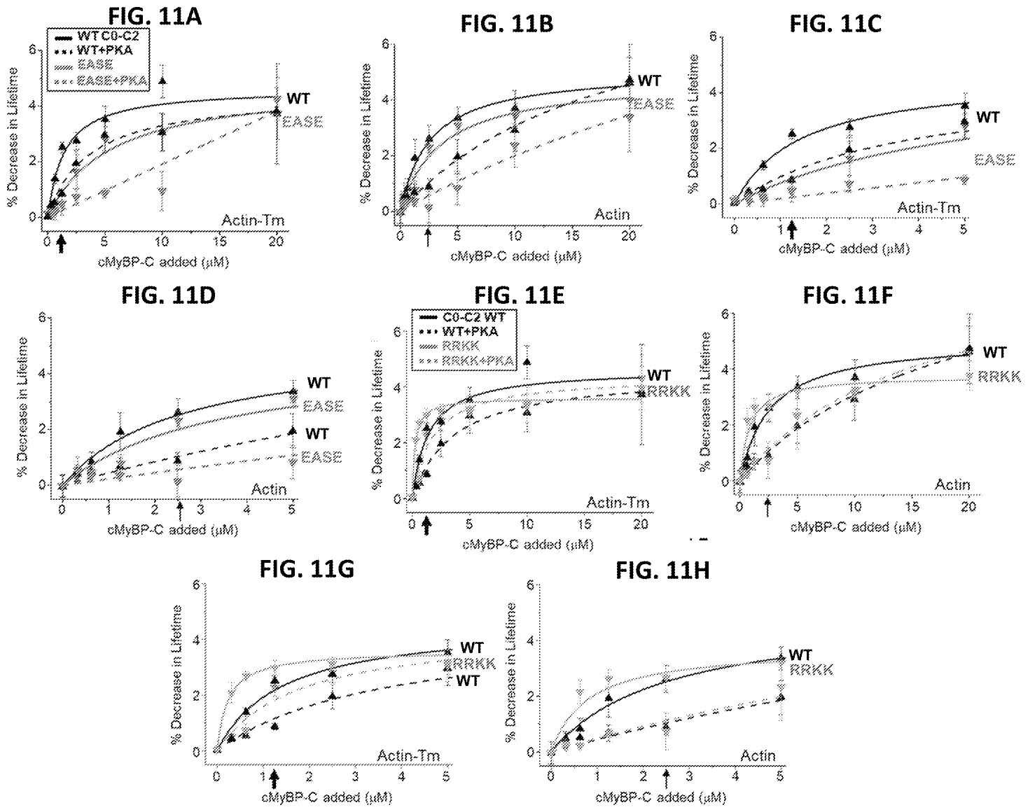

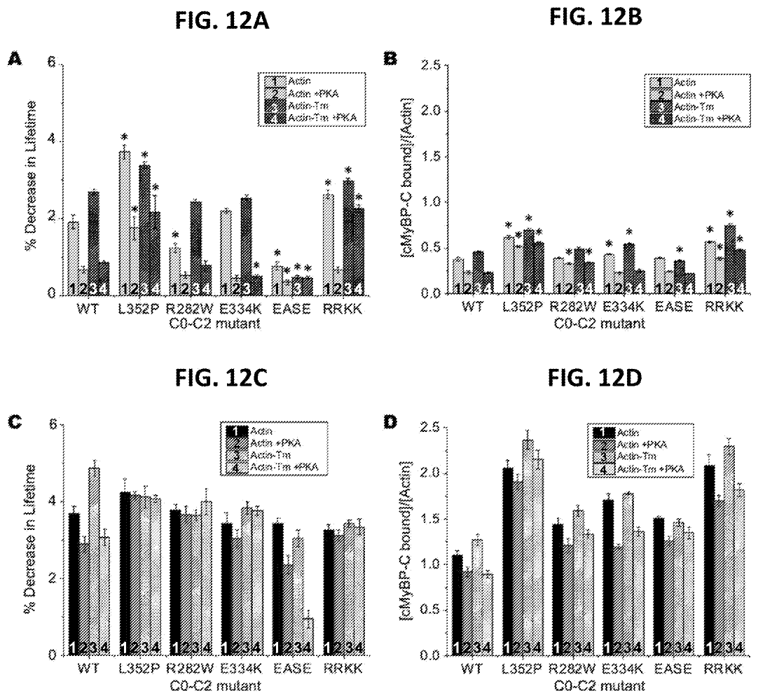

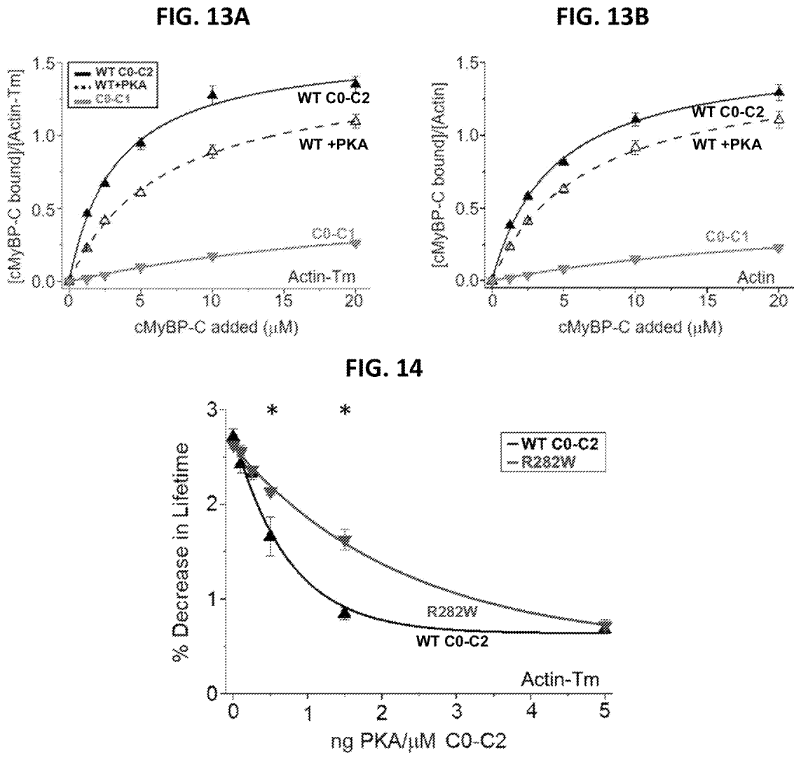

The TR-F actin binding assay in the multi-well format makes feasible the testing of multiple variables. These tests were done at the same time as the individual tests and the results are described herein. Binding curves were determined for the 6 mutant C0-C2 proteins to IAEDANS-actin and IAEDANS-actin-Tm, without and with phosphorylation (FIGS. 10A-10H, and 11A-11H). The apparent Kd and Bmax for the mutants with these variables included are given in Table 4. These curves, though commonly used for comparisons of cMyBP-C to actin, are dependent on maximal binding levels that are subject to artifacts. The maximal levels are also not found in vivo. Therefore, the TR-F results for binding at specific substoichiometric concentrations of C0-C2 have been compared. In most cases, these concentrations are 2.5 μM C0-C2 for actin and 1.25 μM C0-C2 for actin-Tm. They are the same concentrations that showed the largest difference between phosphorylated and non-phosphorylated forms of wild type C0-C2 and they reflect conditions in vivo where C0-C2 would be found only one for every seven actin monomers. In cases where binding is enhanced by mutations (L352P and A216R/S217K), comparisons at lower concentrations were more informative. For comparisons of actin versus actin-Tm when C0-C2 was phosphorylated (and therefore showed reduced binding) comparisons at higher C0-C2 concentrations were most useful. Binding levels, as reported by TR-F, at specific C0-C2 concentrations used for comparison of different conditions are all taken from the binding curves and presented in Table 5 for all mutants. Finally, binding was assessed by cosedimentation for all the mutants at low (submaximal; 1.25 μM) and 8-fold higher (10 μM, to demonstrate that 1.25 μM was indeed submaximal) concentrations. These too were conducted for actin and actin-Tm, unphosphorylated and phosphorylated and can be found in FIG. 12A-12D. For 00-02 and C0-C1 full cosedimentation binding curves were compared (FIG. 13A-13B).

TABLE 4
TR-F and cosedimentation binding parameters
of WT and mutant MyBP-C/actin and actin-Tm.

| | | | | | Cosedimentation | |
|---|---|---|---|---|---|---|
| | | | TR-F | | | Bmax |
| | | | | Bmax (Δτ) | | (Fraction bound; |
| Construct | Filament | PKA | Kd (μM) | (Maximal Δτ) | Kd (μM) | C0-C2/actin) |
| C0-C2 | Actin | − | 2.5 ± 0.4 | 5.1 ± 0.3 | 4.3 ± 0.3 | 1.56 ± 0.04 |
| Wild type | | | | | | |
| | Actin | + | 19 ± 5 | 9 ± 1 | 6.5 ± 0.3 | 1.48 ± 0.03 |
| | Actin-Tm | − | 1.4 ± 0.5 | 4.6 ± 0.4 | 3.3 ± 0.4 | 1.61 ± 0.06 |
| | Actin-Tm | + | 3.6 ± 0.7 | 4.5 ± 0.3 | 6.6 ± 0.4 | 1.46 ± 0.04 |
| C0-C1* | Actin | − | 266 ± 168** | 30 ± 20** | 49 ± 8^{#} | 0.84 ± 0.09^{#} |
| | Actin | + | — | — | — | — |
| | Actin-Tm | − | 49 ± 9^{#} | 9 ± 1^{#} | 23 ± 2^{#} | 0.56 ± 0.03^{#} |
| | Actin-Tm | + | — | — | — | — |
| C0-C2 | Actin | − | 0.7 ± 0.2^{#} | 5.0 ± 0.3 | — | — |
| L352P | | | | | | |
| | Actin | + | 2.1 ± 0.2^{#} | 5.0 ± 0.1^{#} | — | — |
| | Actin-Tm | − | 0.44 ± 0.05^{$} | 4.3 ± 0.1 | — | — |
| | Actin-Tm | + | 1.6 ± 0.2^{#} | 4.6 ± 0.1 | — | — |
| C0-C2 | Actin | − | 2.5 ± 0.9 | 4.8 ± 0.5 | — | — |
| E334K | | | | | | |
| | Actin | + | 20 ± 5 | 4.5 ± 0.4 | — | — |
| | Actin-Tm | − | 1.1 ± 0.3 | 5.7 ± 0.7 | — | — |
| | Actin-Tm | + | 6 ± 2 | 6.5 ± 0.4^{#} | — | — |
| C0-C2 | Actin | − | 3.9 ± 0.4^{#} | 5.2 ± 0.2 | — | — |
| R282W | | | | | | |
| | Actin | + | 14 ± 3 | 8 ± 1 | — | — |
| | Actin-Tm | − | 1.4 ± 0.4 | 4.3 ± 0.3 | — | — |
| | Actin-Tm | + | 8 ± 1^{#} | 4.3 ± 0.3 | — | — |
| C0-C2 | Actin | − | 3.5 ± 0.9 | 4.8 ± 0.4 | — | — |
| R215E/K218E | | | | | | |
| | Actin | + | 57 ± 58** | 13 ± 1**^{#} | — | — |
| | Actin-Tm | − | 5 ± 2^{$$} | 4.9 ± 0.6 | — | — |
| | Actin-Tm | + | ** | ** | — | — |
| C0-C2 | Actin | − | 0.8 ± 0.3^{#} | 3.8 ± 0.3^{#} | — | — |
| A216R/S217K | | | | | | |
| | Actin | + | 16 ± 4 | 8 ± 1 | — | — |
| | Actin-Tm | − | 0.24 ± 0.05^{#} | 3.6 ± 0.1^{#} | — | — |
| | Actin-Tm | + | 1.8 ± 0.5 | 4.4 ± 0.3 | — | — |

In Table 4, data are mean S.E. (n>4) derived from binding curves of 7 concentrations of cMyBP-C (0-20 μM) to 1 μM of actin (see FIGS. 2A-2H, 7-9D, 11A-11H, and 13A-13B) fit to a quadratic equation (see Methods and Materials). *C0-C1 data uses additional 40 μM concentration and all mutant constructs are C0-C2. Data for WT is the same as that presented in Table 1 above and is included here for ease of comparison. See methods for a detailed discussion of K_{d} and Bₘₐₓ uses and limitations. (−) in binding assay data column indicates that the value was not determined as full cosedimentation curves were not done for mutants (FIG. 12 compares cosedimentation and TR-F binding determined at 2 concentrations of C0-C2). Significant difference in mutant C0-C2 as compared to WT under identical conditions, ^{#}p<0.05. Trending towards a difference in mutant C0-C2 as compared to WT under identical conditions, ^{$}p=0.09 or ^{$$}p=0.11. “Does not fit well to quadratic equation (but does lit well to linear function). Kd values were significantly different (p<0.05) between unphosphorylated and phosphorylated WT and mutant C0-C2 in all cases with the only exception being A215E/K218E binding to actin-Tm.

TABLE 5
Change in IAEDANS lifetime for IAEDANS-
Actin and IAEDANS-Actin-Tm upon substoichiometric
binding of C0-C2 and C0-C1.

| cMyBP-C complex with | | | |
|---|---|---|---|
| 1 μM F-actin | | % Decrease in IAEDANS Lifetime | |
| Construct | IAEDANS- | −PKA | +PKA |
| C0-C2 Wild type | Actin | 2.61 ± 0.14 | 0.88 ± 0.08^{&} |
| (2.5 μM) | | | |
| C0-C2 Wild type | Actin | 1.92 ± 0.17 | 0.68 ± 0.08^{&} |
| (1.25 μM) | | | |
| C0-C2 Wild type | Actin | 0.83 ± 0.10 | 0.5 ± 0.06^{&} |
| (0.625 μM) | | | |
| C0-C2 Wild type | Actin-Tm | 2.81 ± 0.10 | 1.90 ± 0.10^{&@} |
| (2.5 μM) | | | |
| C0-C2 Wild type | Actin-Tm | 2.70 ± 0.07^{@} | 0.71 ± 0.06^{&@} |
| (1.25 μM) | | | |
| C0-C2 Wild type | Actin-Tm | 1.70 ± 0.12^{@} | 0.42 ± 0.05^{&} |
| (0.625 μM) | | | |
| C0-C1 (Δm-C2) | Actin | 0.17 ± 0.06^{#} | — |
| (2.5 μM) | | | |
| C0-C1 (Δm-C2) | Actin-Tm | 0.53 ± 0.05^{#+} | — |
| (2.5 μM) | | | |
| C0-C2 L352P | Actin | 3.62 ± 0.19^{#} | 2.80 ± 0.16^{#&} |
| (2.5 μM) | | | |
| C0-C2 L352P | Actin | 3.73 ± 0.19^{#} | 1.76 ± 0.30^{#&} |
| (1.25 μM) | | | |
| C0-C2 L352P | Actin-Tm | 3.39 ± 0.06^{#¥} | 2.17 ± 0.29^{#&} |
| (1.25 μM) | | | |
| C0-C2 E334K | Actin | 2.79 ± 0.11 | 0.72 ± 0.09^{&} |
| (2.5 μM) | | | |
| C0-C2 E334K | Actin | 2.20 ± 0.06 | 0.45 ± 0.07^{$&} |
| (1.25 μM) | | | |
| C0-C2 E334K | Actin | 0.46 ± 0.03^{#} | 0.19 ± 0.05^{#&} |
| (0.625 μM) | | | |
| C0-C2 E334K | Actin | 0.25 ± 0.03^{$} | 0.40 ± 0.03^{&} |
| (0.313 μM) | | | |
| C0-C2 E334K | Actin-Tm | 2.90 ± 0.07 | 2.16 ± 0.06*^{&@} |
| (2.5 μM) | | | |
| C0-C2 E334K | Actin-Tm | 2.52 ± 0.10⁺ | 0.50 ± 0.04^{#&} |
| (1.25 μM) | | | |
| C0-C2 E334K | Actin-Tm | 1.95 ± 0.06^{$@} | 0.36 ± 0.06^{&+} |
| (0.625 μM) | | | |
| C0-C2 E334K | Actin-Tm | 0.97 ± 0.09^{#@} | 1.37 ± 0.08^{&} |
| (0.313 μM) | | | |

Substoichiometric binding levels (for wild type and mutant C0-C2) on 1 μM IAEDANS-labeled actin and actin-Tm. Data points shown are taken from the binding curves in FIGS. 2A-2H, 7-9D, and 10A-11H For comparison of mutants with wild type *p<0.05 and #p<0.01 or trending differences 0.05<$p<0.12. For effects of PKA phosphorylation \hat{ }p<0.05 and &p<0.01 or trending differences 0.05<€p<0.12. For Tm effects, +p<0.05 and @p<0.01 or trending differences 0.05<¥p<0.12. Data is provided as mean±SE (n>4).

C0-C1 decreases actin binding. Both the M-domain and the C2 domain provide binding sites for actin and their removal, in C0-C1, dramatically reduces acting binding and this is seen in the TR-F assay as well (FIG. 7, Table 2, >90% reduction in binding at 2.5 μM, p<0.0001)

C0-C1 binding to actin is increased by Tm. Though C0-C1 binding to actin is reduced compared to C0-C2 (see above), its binding is increased by the presence of Tm. Comparing binding at 2.5 μM C0-C1, binding to actin-Tm was increased by 210% (p<0.05). As the motif (containing the PKA phosphorylation sites) is absent in C0-C1, PKA treated C0-C1 was not done. Cosedimentation results under identical conditions show similar findings (Table 4 and FIG. 13A-13B).

L352P increase binding. The TR-F assay readily detected the increase in binding of L352P to actin (FIG. 7 and Table 2). Increases of 39% were seen at 2.5 μM L352P C0-C2 (p<0.0005). The TR-F assay readily detected a 26% increase in binding of 1.25 μM C0-C2 L352P versus wild type (p<0.00001) to actin-Tm. When phosphorylated by PKA, the increases in binding of L352P to actin (at 2.5 μM C0-C2) and actin-Tm (at 1.25 μM C0-C2) were 218% (p<0.005) and 204% (p<0.00001), respectively, relative to WT C0-C2. At the same concentrations, phosphorylation of L352P reduced its binding to actin, compared to unphosphorylated L352P, by 53% (p<0.0001) and its binding to actin-Tm by 36% (p<0.0001). The effect of Tm on the binding of L352P, either unphosphorylated or phosphorylated, was not significant. Cosedimentation measurement of L352P, at submaximal binding levels, to actin and actin-Tm showed qualitatively the same effects as reported by TR-F. Binding was increased by L352P in both the unphosphorylated and phosphorylated states to both actin and actin-Tm (all >80% and p<0.000001).

E334K does not alter actin binding, but increases actin-Tm binding. The HCM mutant, E334K, was tested because it significantly reduced actin binding when present in mouse C1-C2 and the homologous mutant (E330K) in mouse hearts shortened the duration of ejection. However, E334K in human C0-C2 displayed binding that was similar to wild type measured by TR-F (FIG. 7 and Table 2). The E334K mutation did not show significant changes in binding to actin at 2.5 μM nor to actin-Tm at 1.25 μM when compared with wild type C0-C2. Comparing phosphorylated E334K and wild type showed no significant differences in binding to actin or actin-Tm (FIG. 10A-10H). Phosphorylation of E334K reduced its binding to actin (at 2.5 μM) by 74% (p<0.0001) and its binding to actin-Tm (at 1.25 μM) by 80% (p<0.0001). Tm increased E334K's binding compared to actin alone by approximately 300% at 0.313 and 0.625 μM E334K (p=0.011). For phosphorylated E334K at 2.5 μM, Tm increased binding by 200% (p<0.0001). In cosedimentation measurements of E334K at 1.25 μM, binding to actin and actin-Tm was observed to be modestly increased (14% (p<0.02) and 19% (p<0.0001), respectively). No significant changes were observed with phosphorylated E334K.

R282W alters phosphorylation and actin binding. R282 changes the PKA target sequence (281-284) from RRIS to RWIS (FIG. 6A-6C) and this change is predicted to eliminate PKA phosphorylation of Ser284. Ser284 is one of four serines in the M-domain phosphorylated by PKA. Without wishing to limit the present invention to any theories or mechanisms it was predicted that R282W would eliminate PKA phosphorylation of this serine. Under standard conditions R282W showed subtle, but not significant differences in binding, when compared to wild type in either the phosphorylated or unphosphorylated state at 2.5 μM. At 1.25 μM the unphosphorylated R282W did display a significant decrease in binding (36% decrease, p<0.01) (FIG. 8A-8D and Table 2). On actin-Tm R282W again showed a reduction in binding of 10% at 1.25 μM (p<0.01) compared to wild type. The same reduced PKA effects (more binding and larger change in TR-F) was observed for binding to actin-Tm when R282W was treated with intermediate levels of PKA (0.5 and 1.5 ng PKA/μM C0-C2) (FIG. 14). At high levels of phosphorylation mediated by 7.5 ng PKA/μg C0-C2, R282W binding to actin-Tm was the same as wild type. As was the case for wild type, maximal phosphorylation of R282W significantly reduced its binding to actin by 68% (p<0.0001) and actin-Tm by 68% (p<0.0001). Tm increased the binding of unphosphorylated R282W, at 1.25 μM, by 97% to actin (p<0.0001) and phosphorylated R282W, at 2.5 μM, by 104% (p<0.005). Cosedimentation showed similar levels of binding of R282W, compared to wild type C0-C2, to actin and actin-Tm when unphosphorylated but increased binding to actin (39%, p<0.002) and actin-Tm (48%, p<0.002) when R282W was maximally phosphorylated.

Examination by mass spectrometry of the 4 serines (Ser275, Ser284, Ser304, and Ser311) in the motif that are phosphorylated by PKA showed that, at PKA levels required for maximal phosphorylation (2.5 ng PKA/μg C0-C2) or 25× those levels, all 4 serines were phosphorylated in wild type C0-C2. In contrast, in the R282W mutant phosphorylation was almost undetectable at Ser284. Phosphorylation was detected in the mutant at Ser275, Ser304, and Ser311 (Tables 6 and 7). The initial test of PKA effects on actin binding utilized PKA levels 3× that required for maximal phosphorylation. From these results it was concluded that phosphorylation in some combination of Ser275, Ser304, and Ser311 in R282W is sufficient to reduce interactions with actin.

TABLE 6
WT and R282W C0-C2 phosphorylated peptides.

| SEQ | | | Precursor | Ob- | Site |
|---|---|---|---|---|---|
| ID | | | charge | served | Score |
| NO: | Peptide | Site | (z) | m/z | (Ascore) |
| 2 | R.TS#LA | 275 | 2 | 399.7 | 1000 |
| | GGGR.R | | | | |
| 3 | R.RTS#L | 275 | 2 | 477.7 | 24.4 |
| | AGGGR.R | | | | |
| 4 | R.RIS#D | 284 | 3 | 738.4 | 30.8 |
| | SHEDTGI | | | | |
| | LDFSSLL | | | | |
| | K.K | | | | |
| 5 | Mutant | 284 | 3 | 748.3 | 6.6 |
| | R.WIS#D | | | | |
| | SHEDTGI | | | | |
| | LDFSSLL | | | | |
| | K.K | | | | |
| 6 | K.KRDS# | 304 | 2 | 444.7 | 1000 |
| | FR.T | | | | |
| 7 | K.RDS#F | 304 | 2 | 380.7 | 1000 |
| | R.T | | | | |
| 8 | K.RDS#F | 304 | 3 | 414.9 | 23.6 |
| | RTPR.D | | | | |
| 9 | R.DS#KL | 311 | 3 | 694.0 | 1000 |
| | EAPAEED | | | | |
| | VWEILR.Q | | | | |

In Table 6, LC-MS/MS data from all identified C0-C2 phosphopeptide sequences, where S #is the phosphorylated Ser of the peptide in C0-C2 samples treated with PKA. The dots (.) indicate cleavage sites, where the protease cleaves between the two residues separated by the dot, and thus the actual peptides do not contain the extra residue outside of the dot. Site score values ≥19 are considered confidently assigned with near certainty (>99%) and values listed as 1000 denotes that there are no other possible sites to assign the phosphate and the phosphoserine assignment within the peptide is 100% unequivocal. A low site score for the Mutant phosphorylated peptide is likely due to its almost complete absence (see Table 7) and the presence of additional serines/threonines in the peptide, not allowing for it to be confidently assigned. Mutant unphosphorylated peptide was observed (z=3, m/z=741.7) and used to calculate a ratio of peak intensities of phosphorylated to unphosphorylated peptide in Table 7.

TABLE 7
Peak intensity ratios for WT and R282W PKA
phosphorylated:unphosphorylated peptides.

| | Ser275 | Ser284 | Ser304 | Ser311 |
|---|---|---|---|---|
| C0-C2 | S#/S | S#/S | S#/S | S#/S |
| WT | 1.52 | 2.46 | 30.49 | 0.012 |
| R282W | 1.95 | <0.0005 | 41.72 | 0.012 |

Table 7 shows mass spectrometry intensity ratios, S #/S, of peptides for each PKA site (Ser275, Ser284, Ser304, and Ser311). S is the intensity of the unphosphorylated peptide peak and S #is the intensity of the phosphorylated peptide peak in the PKA-treated sample as by detected mass spectrometry. This data is from treatment of C0-C2 with 2.5 ng PKA/μg C0-C2. Data from treatment with 25× this amount (66 ng PKA/μg C0-C2) showed no increase in the S #/S for Ser284 in the R282W mutant protein.

In vivo, the PKA target serines are not found completely phosphorylated. Therefore, a range of phosphorylation levels of C0-C2 were tested for their effects on binding to actin. As for testing other conditions, use of the TR-F assay in a 384-well plate format made this relatively easy. The PKA levels used were reduced to phosphorylate wild type and R282W C0-C2 for actin binding studies. The effect of submaximal phosphorylation on the change in TR-F upon binding to actin was measured. Immediately following determination of the TR-F, a portion of the samples was removed with a multi-channel pipette and placed in a protein sample buffer, run on SDS-PAGE gels, and stained with Pro-Q Diamond to determine phosphorylation levels. A wide range of phosphorylation levels was obtained by varying PKA levels and the R282W mutant was less phosphorylated than wild type at all PKA concentrations (FIG. 8B-8C). At levels of phosphorylation mediated by PKA at 1.5 ng PKA/μg C0-C2, binding of wild type C0-C2 remained low—at the same level as that seen when either it or the R282W were treated with 5 ng PKA/μg C0-C2. R282W, at this concentration (1.5 ng PKA/μg C0-C2), showed a 65% increase in binding to actin (FIG. 8D, p<0.00001). As PKA levels were reduced further to 0.5 ng PKA/μg C0-C2, the difference between wild type and the mutant remained significant (FIG. 5D, 27%, p=0.0009), though both now exhibited increased binding.

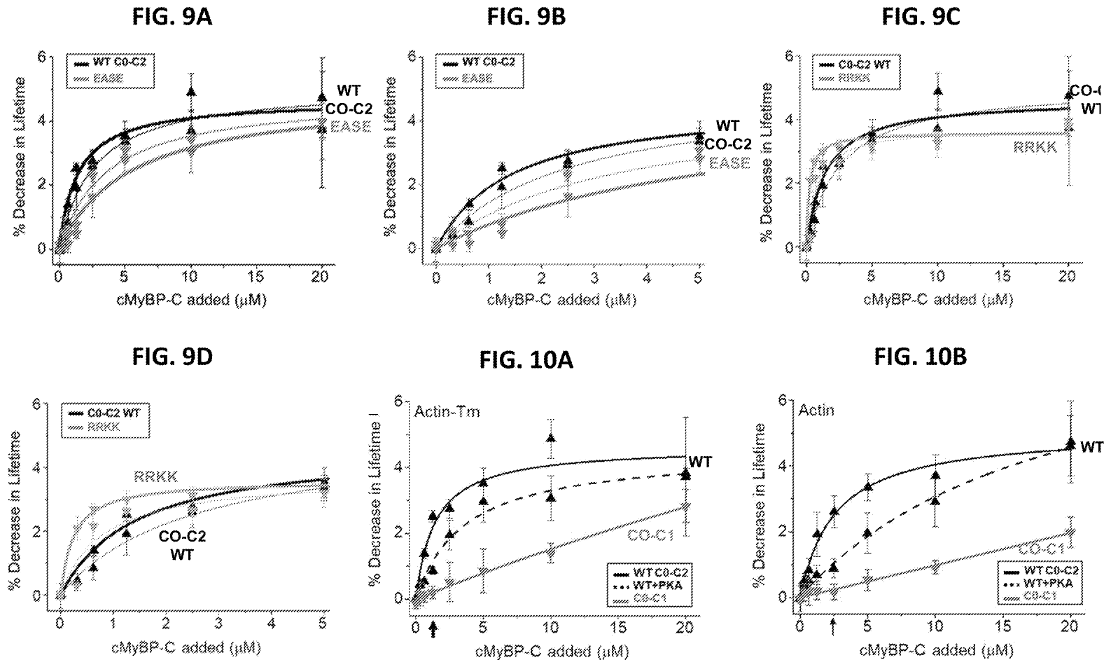

Mutations in the RASK sequence of C1 affect actin-Tm binding. Binding to actin by C0-C2 is increased when actin is decorated with Tm (FIGS. 2C and 2F, Table 1). To specifically investigate the ability of the TR-F screen to detect changes in cMyBP-C interactions with tropomyosin, functional mutations in the C1 domain were made in a region (amino acids 215-218; RASK) that interacts with tropomyosin. Mutating this sequence to EASE (R215E/K218E; changing the positively charged R and K to the negatively charged E) resulted in 82% lower levels of binding to actin-Tm at 1.25 μM when compared to wild type (p<1×10¹³). This mutant, EASE, displayed 37% lower levels of binding at 1.25 μM C0-C2 to actin-Tm than to actin alone (p=0.026) (FIG. 9A-9B, Table 2). This is the only mutation tested that resulted in less binding to actin-Tm than to actin. In contrast, wild type C0-C2 shows a 41% greater TR-F effect on actin-Tm than on actin alone (p=0.003) (FIG. 2A-2F, and repeated in FIG. 9A-9D, Table 2). Addition of positively charged amino acids to the Tm binding region, RRKK (A216R/S217K) enhanced binding at 1.25 μM C0-C2 compared with wild type to actin-Tm by 10% (p=0.021). At lower concentrations of 0.625 and 0.313 μM these relative differences were even greater, being 58% and 275%, respectively (p<1×10⁻⁶) (FIG. 9D, Table 2). On actin alone, at lower concentrations, 1.25 and 0.625 μM C0-C2, the RRKK increased binding by 37% and 160% compared with wild type (p<0.006; FIG. 9D and Table 2). The negative (EASE) mutation had negative effects on binding to bare actin as well. Binding was reduced by 60% (p<0.001) at 1.25 μM C0-C2 when compared to wild type on actin but these effects are reduced compared to those with actin-Tm (FIG. 9B and Table 2).

RASK (amino acids 215-218, in C1 that interact with Tm) mutations were shown to reduce actin-Tm binding upon charge reversal (RASK>EASE) or enhance actin-Tm binding with added positively charged amino acids (RASK>RRKK). Smaller changes in binding to bare actin were also observed. These mutants compared to wild type continued to show differences when phosphorylated.

RASK>EASE when phosphorylated, showed 62% reduction compared to wild type (p<0.00001) in binding to actin-Tm, at 2.5 μM. On bare actin, phosphorylated RASK>EASE compared to wild type showed a 43% reduction (p<0.03). Phosphorylation further reduced binding of the EASE mutant, at 2.5 μM, to actin by 78% (p<0.00001) and 56% on actin-Tm (p<0.001). As mentioned in above, compared to actin alone Tm reduced binding of unphosphorylated RASK>EASE by 37% (p<0.03). For phosphorylated RASK>EASE Tm neither enhances nor reduces binding (FIG. 11A-11H). Cosedimentation assays showed RASK>EASE reduced binding to actin-Tm by 22% (p<0.00001). No significant changes were observed in binding to actin or when EASE was phosphorylated.

RASK>RRKK (at 1.25 μM) when phosphorylated, showed a 217% increase (p<0.00001) in binding to actin-Tm when compared with wild type. Phosphorylated RASK>RRKK compared to wild type on bare actin showed no significant difference in binding. This illustrates the importance of the RASK sequence to Tm interactions over actin interactions. Phosphorylation reduced binding of the RRKK mutant, at 1.25 μM, to actin by 75% (p<0.00001) and at 0.625 μM, to actin-Tm by 73% (p<0.00001). Tm increased binding of unphosphorylated RASK>RRKK by almost 600% (p<0.000001) over that seen for actin alone at 0.313 μM. For phosphorylated RASK>RRKK Tm enhances binding by 274% (p<0.000001) over that seen for actin alone at 2.5 μM. For comparison, Tm enhances binding of unphosphorylated wild type (0.625 μM) by 105% (p<0.000001) and for phosphorylated wild type (2.5 μM) by 115% (p<0.000001) over that seen for actin alone. Cosedimentation assays showed RASK>RRKK at 1.25 μM increased binding when it was unphosphorylated or phosphorylated. For unphosphorylated RASK>RRKK the increase over wild type was 51% and for actin-Tm it was 61%. For phosphorylated RASK>RRKK the increase over wild type was 65% and for actin-Tm it was 108% (p<0.0001 for all of these comparisons).

Novel findings comprise: (1) Binding of cMyBP-C N-terminal domains, C0-C2, causes changes in the lifetime of F-actin labeled at Cys374 with all 5 fluorescent dyes tested; (2) IAEDANS-labeled actin and IAEDANS-actin-tropomyosin (Tm) gave the largest and most consistent changes in lifetime upon binding; (3) The TR-F assay is capable of distinguishing between binding of unphosphorylated and phosphorylated C0-C2; (4) For suitability in high-throughput screening (HTS), TR-F assay quality for comparisons of sample conditions (i.e., unbound vs. bound actin and unphosphorylated vs. phosphorylated C0-C2) was determined to be good to excellent as indexed by the Z′ score; (5) The TR-F assay has adequate sensitivity to detect changes in cMyBP-C binding due to 2 (of the 3 tested) hypertrophic cardiomyopathy (HCM) disease mutations and removal of the M-domain and C2. Results from parallel cosedimentation experiments confirmed TR-F results for all the constructs; (6) The HCM mutant R282W, which disrupts the recognition sequence of one of the three PKA sites in cMyBP-C, eliminates phosphorylation of one serine and reduces phosphorylation of the remaining sites as shown by mass spectrometry; (7) Reduced phosphorylation in R282W leads to changes in binding to actin and actin-Tm, as shown by the TR-F assay, suggesting that this may be the cause of the HCM phenotype; (8) The TR-F assay is capable of monitoring Tm as well as actin interactions. This was illustrated by Tm-binding mutations, in a proposed Tm binding sequence in C1, showing that reversal of positively charged residues to negatively charged residues reduced binding to IAEDANS-actin-Tm, whereas addition of more positive charge increased binding.

Without wishing to limit the present invention, together these findings suggest that the TR-F assay is suitable for rapidly and accurately determining quantitative binding and useful for experiments of screening of physiological conditions or compounds that affect cMyBP-C binding to actin filaments. The present invention features time-resolved fluorescence (TR-F) to monitor protein binding and the effects of phosphorylation and mutations on the binding, for example, monitor binding of the N-terminal domain of cMyBP-C (C0-C2) to actin and actin-Tm.

The TR-F assay based on IAEDANS-labeled F-actin, in the 384-well plate format, has proven conducive to quick and easy optimization of conditions for binding of cMyBP-C C0-C2 to actin and actin-Tm. Testing the binding of multiple mutant versions of C0-C2, and distinguishing between binding by phosphorylated and unphosphorylated states indicates it can be used as a high-throughput complementary assay to current cosedimentation assays that are much more labor intensive. Z′ score analysis indicates the assay is suitable for screening for drugs that modulate cMyBP-C binding to actin or those that mimic the effects of phosphorylation on binding. In the absence of other screens with these specific capacities at the high-throughput level, the TR-F assay described here is a major advancement toward drug development based on cMyBP-C activity

The present invention features a novel fluorescence lifetime-based assay to identify small-molecule inhibitors of actin-MyBP-C binding. The method comprises labeling actin with a fluorescent dye (Alexa Fluor 568, 568) near its cMyBP-C binding sites. When combined with cMyBP-C N-terminal fragment, C0-C2, the fluorescence lifetime of 568-actin decreases. Using this reduction in lifetime as a readout of actin binding, a high-throughput screen of a 1280-compound library identified 3 compounds that reduced C0-C2 binding to actin in the micromolar range. Binding of phosphorylated C0-C2 was also blocked by these compounds. That they specifically block binding was confirmed by a novel actin-C0-C2 time-resolved FRET (TR-FRET) binding assay. Isothermal titration calorimetry (ITC) and transient phosphorescence anisotropy (TPA) confirmed that the compounds bind to cMyBP-C but not actin. TPA results were also consistent with the drugs inhibiting C0-C2 binding to actin. The actin-cMyBP-C lifetime assay permits detection of pharmacologically active compounds that affect actin-cMyBP-C function. TPA, TR-FRET, and ITC can then be used to understand the mechanism by which the compounds alter cMyBP-C interactions with actin.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

1. A method of using time-resolved fluorescence (TR-F) and a fluorescent protein biosensor to quantitate protein binding in solution, the method comprising:
a) labelling (operably connecting) a first protein with a fluorescent probe to generate a fluorescent protein biosensor suitable for TR-F;
b) contacting including binding the first protein that is operably connected/labelled to the fluorescent probe to a second protein in solution;
c) measuring TR-F fluorescence lifetime when the first protein contacts a second protein effectuating fluorescence, wherein fluorescence lifetime is the time that a fluorescent probe is in the excited state and decays down to the ground state; and
d) quantitating protein contact including binding using the measured fluorescence lifetime.

2. (canceled)

3. The method of claim 1, wherein a physiological change/perturbation, phosphorylation, and/or mutation of the second protein in proximity of operable connection between the first protein and second protein affects a change in contact including binding of first protein affecting/changing fluorescence lifetime, wherein change in fluorescence lifetime quantitates the change in contact including binding.

4. The method of claim 1, wherein the first protein comprises actin, globular actin (G-actin), fibrous-actin (F-actin), actin filament, actin-tropomyosin complex, tropomyosin (Tm), and the regulated thin filament.

5. The method of claim 1, wherein the second protein comprises cardiac myosin binding protein-C (cMyBP-C), skeletal MyBP-C, and fragments thereof (e.g., C0-C2).

6. The method of claim 1, wherein the fluorescent probe is selected from a group consisting of IAEDANS, IAANS, CPM, IANBD, 5-IAF, FMAL, Alexa Fluor 488, Alexa Fluor 532, and Alexa Fluor 568.

7. The method of claim 1, wherein the method detects binding properties of the first protein by correlating time-resolved fluorescence lifetime with protein binding interactions in solution.

8. The method of claim 1, wherein the method is for screening physiological conditions or compounds that affect the second protein contact including binding to the first protein.

9. A method of identifying a molecule that modulates actin binding to actin-binding proteins (ABPs) comprising:
a) labelling (operably connecting) actin protein with a fluorescent probe suitable for time-resolved fluorescence (TR-F);
b) contacting including binding actin that is operably connected/labelled to the fluorescent probe to an actin binding protein (ABP) in solution;
c) measuring TR-F fluorescence lifetime when fluorescent probe labelled-actin contacts or binds ABP in the presence and absence of a molecule, wherein fluorescence lifetime is the time that a fluorescent probe is in the excited state and decays down to the ground state;
d) quantitating protein binding using the measured fluorescence lifetime in the presence and absence of molecule; and
e) identifying the molecule as a modulator of actin-ABP binding when a change occurs in TR-F fluorescence lifetime in presence of the molecule as compared to absence of the molecule.

10. (canceled)

11. The method of claim 9, wherein a physiological change/perturbation, phosphorylation, and/or mutation of the ABP in proximity of operable connection between actin and ABP affects a change in binding of actin affecting/changing fluorescence lifetime, wherein change in fluorescence lifetime quantitates the change in binding.

12. The method of claim 9, wherein actin comprises globular actin (G-actin), fibrous-actin (F-actin), actin filament, actin-tropomyosin complex, or thin filaments.

13. The method of claim 9, wherein the ABP comprises cardiac myosin binding protein-C (cMyBP-C), skeletal MyBP-C or MyBP-C fragments (e.g., C0-C2).

14. The method of claim 9, wherein the fluorescent probe is selected from a group consisting of IAEDANS, IAANS, CPM, IANBD, 5-IAF, FMAL, Alexa Fluor 488, Alexa Fluor 532, and Alexa Fluor 568.

15. The method of claim 9, wherein the method detects binding properties of actin by correlating time-resolved fluorescence lifetime with actin-ABP binding interactions in solution.

16. The method of claim 9, wherein the method is for screening physiological conditions or compounds that affect the ABP binding to actin.

17. (canceled)

18. The method of claim 9, wherein the method is for screening or identifying physiological conditions or compounds that affect cardiac myosin binding protein-C (cMyBP-C) binding to actin, actin filaments, and/or actin-tropomyosin complexes.

19. The method of claim 9, wherein the method is a TR-F-based screen that detects changes in actin binding brought about by phosphorylation of the cMyBP-C N-terminal C0-C2 fragment.

20. The method of claim 9, wherein the method is for high throughput drug discovery or screening assay to identify drugs that mimic phosphorylation by inducing similar changes in binding that reduce actin-cMyBP-C binding with increased phosphorylation or by mutations (that reduce/decrease binding), or that enhance actin-cMyBP-C binding with decreased phosphorylation or by mutations (that enhance/increase binding) and measuring lifetime fluorescence change of phosphorylated-induced protein binding.

21. The method of claim 9, wherein the method is for mimicking phosphorylated state of cMyBP-C.

22. The method of claim 9, wherein the method is for screening drugs that modulate cMyBP-C (in either the phosphorylated or non-phosphorylated state) binding to actin-Tm.

23. A method of identifying a test compound that modulates actin binding to actin-binding proteins (ABPs) or identifying a test compound that modulates an actin+actin-binding protein (ABP) complex or its microenvironment, wherein the method is suitable for high throughput screening (HTS), the method comprising:
a) providing actin with a fluorescent probe suitable for time-resolved fluorescence (TR-F);
b) introducing actin with the fluorescent probe suitable for TR-F to an actin binding protein (ABP) in solution;
c) measuring TR-F fluorescence lifetime when actin with the fluorescent probe suitable for TR-F contacts or binds ABP in the presence and absence of a test compound, wherein fluorescence lifetime is the time that a fluorescent probe is in the excited state and decays down to the ground state;
d) quantitating protein binding using the measured fluorescence lifetime in the presence and absence of the test compound; and
e) identifying the test compound as a modulator of actin-ABP binding when a change occurs in TR-F fluorescence lifetime in presence of the test compound as compared to absence of the test compound.

24. (canceled)


SEQUENCE LISTING (9 sequences)

SEQ ID NO: 1 - protein, 105 aa, Homo sapiens
AMGTGDLDLLSAFRRTSLAGGGRRISDSHEDTGILFSSLLKKRDSFRTPRDSKLEAPAEEDVWEILRQAPPSEYERIAFGYGVTDLRGMLKRLKGMRRDEKKSTA

SEQ ID NO: 2 - protein, 10 aa, Homo sapiens
  MOD_RES (3)..(3): PHOSPHORYLATION
RTSLAGGGRR

SEQ ID NO: 3 - protein, 11 aa, Homo sapiens
  MOD_RES (4)..(4): PHOSPHORYLATION
RRTSLAGGGRR

SEQ ID NO: 4 - protein, 21 aa, Homo sapiens
  MOD_RES (4)..(4): PHOSPHORYLATION
RRISDSHEDTGILDFSSLLKK

SEQ ID NO: 5 - protein, 21 aa, Homo sapiens
  MOD_RES (4)..(4): PHOSPHORYLATION
RWISDSHEDTGILDFSSLLKK

SEQ ID NO: 6 - protein, 8 aa, Homo sapiens
  MOD_RES (5)..(5): PHOSPHORYLATION
KKRDSFRT

SEQ ID NO: 7 - protein, 7 aa, Homo sapiens
  MOD_RES (4)..(4): PHOSPHORYLATION
KRDSFRT

SEQ ID NO: 8 - protein, 10 aa, Homo sapiens
  MOD_RES (4)..(4): PHOSPHORYLATION
KRDSFRTPRD

SEQ ID NO: 9 - protein, 19 aa, Homo sapiens
  MOD_RES (3)..(3): PHOSPHORYLATION
RDSKLEAPAEEDVWEILRQ